(19) United States
(12) Patent Application Publication
(10) Pub. No.: US 2010/0080855 A1
(43) Pub. Date: Apr. 1, 2010

(54) HEDGEHOG SIGNALING AND CANCER STEM CELLS IN HEMATOPOIETIC CELL MALIGNANCIES

(75) Inventors: Philip A. Beachy, Stanford, CA (US); Tannishtha Reya, Chapel Hill, NC (US)

Correspondence Address: DLA PIPER LLP (US), 4365 EXECUTIVE DRIVE, SUITE 1100, SAN DIEGO, CA, 92121-2133, US

(73) Assignees: THE BOARD OF TRUSTEES OF THE LELAND STANFORD JUNIOR UNIVERSITY, Palo Alto, CA (US); DUKE UNIVERSITY, Durham, NC (US)

(21) Appl. No.: 12/558,429

(22) Filed: Sep. 11, 2009

Related U.S. Application Data
(60) Provisional application No. 61/096,628, filed on Sep. 12, 2008.

(51) Int. Cl. A61K 33/36; A61K 31/343; A61K 31/497; A61K 31/506

(52) U.S. Cl. 424/623; 514/462; 514/252.18; 514/252.19; 514/275

(57) ABSTRACT

The present disclosure relates generally to methods of treating hematopoietic cell malignancy in a subject by administering to the subject a Hedgehog (Hh) pathway antagonist, such as a Smoothen (Smo) antagonist alone or in combination with an ABL-kinase antagonist. Specifically, the disclosure relates to a method of treating hematopoietic cell malignancy in a subject by administering the Hh pathway antagonist, such as the Smo antagonist cyclopamine, in combination with the ABL-kinase antagonist imatinib.

### RELATED APPLICATIONS

This application claims priority under 35 U.S.C. §119 to U.S. Provisional Patent Application No. 61/096,628 filed Sep. 12, 2008 the entire content of which is herein incorporated by reference.

### FIELD OF THE INVENTION

The present invention relates generally to methods of treating hematopoietic cell malignancies and more specifically to use of a Hedgehog (Hh) pathway antagonist, such as a Smoothened (Smo) antagonist, alone or in combination with an BCR-ABL antagonist to treat hematopoietic diseases.

### BACKGROUND OF THE INVENTION

Hedgehog (Hh) signaling pathway is known to play an important role in embryonic development, but the function of Hh signaling in tissue renewal and maintenance is unclear. Importantly, the role of the Hh signaling pathway in malignancies, such as cancers, is essentially unknown.

The Hh signaling pathway is presently understood to function according to the following general model. First, ligands such as the Sonic hedgehog (Shh), Indian hedgehog (Ihh) and Desert hedgehog (Dhh) are produced by cells and bind to the transmembrane receptor protein Patched (Ptch). The Ptch protein is a seven-transmembrane receptor protein that normally binds and inhibits the Smoothened protein (Smo) in the absence of these ligands. Second, Smo because activated when the Ptch protein binds a ligand. Smo is another seven-transmembrane receptor protein. Finally, activated Smo mediates the transcriptional activation of Hh pathway targets via the Glioblastoma (Gli) family of transcriptional factors.

Chronic myelogenous leukemia (CML) is a hematopoietic cell malignancy. CML arises as a consequence of a translocation that fuses the BCR (breakpoint cluster region) serine/threonine kinase gene with the ABL tyrosine kinase. The resulting oncogenic fusion protein is called BCR-ABL and is a cause of CML.

Imatinib is a tyrosine kinase inhibitor used to treat CML patients. Imatinib binds the ABL kinase domain of the BCR-ABL fusion protein and inhibits phosphorylation of downstream substrates to help control CML. Thus, imatinib is a BCR-ABL antagonist. Unfortunately, the cancerous cells responsible for CML can become resistant to imatinib treatment due to mutations in the imatinib binding site of the BCR-ABL fusion protein. Imatinib drug resistance frequently occurs in the advanced stages of CML. The development of imatinib drug resistance in individuals with CML that was previously sensitive to imatinib treatment can lead to relapse and CML disease progression.

CML disease progression may also be driven by cancer stem cells which can propagate CML. One mechanism by which cancer stem cells propagate CML is believed to involve the production of new leukemic cells. Importantly, such new leukemic cells may be imatinib resistant. Altogether, this means that identifying signals that may be required for CML disease progression in general, and CML cancer stem cell maintenance in particular, has become of critical importance as a step toward designing new therapies.

Thus, a need exists for therapies that effectively treat hematopoietic cell malignancies, such as CML and imatinib resistant CML.

### SUMMARY OF THE INVENTION

The present invention relates generally to methods of treating hematopoietic cell malignancy in a subject by administering to the subject a Hedgehog (Hh) pathway antagonist, such as a Smo antagonist, alone or in combination with a BCR-ABL antagonist.

One aspect of the disclosure is a method of treating a hematopoietic cell malignancy in a subject comprising administering a therapeutically effective amount of an Hh antagonist to a subject with a hematopoietic cell malignancy; whereby the hematopoietic cell malignancy is treated.

Another aspect of the disclosure is a method of treating chronic myelogenous leukemia in a subject comprising administering a therapeutically effective amount of an Hh antagonist to a subject with chronic myelogenous leukemia; whereby the chronic myelogenous leukemia is treated.

Another aspect of the disclosure is a method of treating chronic myelogenous leukemia in a subject comprising administering a therapeutically effective amount of a Smo antagonist to a subject with chronic myelogenous leukemia; whereby the chronic myelogenous leukemia is treated.

Another aspect of the disclosure is a method of treating a hematopoietic cell malignancy in a subject comprising administering a therapeutically effective amount of an Hh antagonist and a therapeutically effective amount of a BCR-ABL antagonist to a subject with a hematopoietic cell malignancy; whereby the hematopoietic cell malignancy is treated.

Another aspect of the disclosure is a method of treating chronic myelogenous leukemia in a subject comprising administering a therapeutically effective amount of an Hh antagonist and a therapeutically effective amount of a BCR-ABL antagonist to a subject with chronic myelogenous leukemia; whereby the chronic myelogenous leukemia is treated.

Another aspect of the disclosure is a method of treating chronic myelogenous leukemia in a subject comprising administering a therapeutically effective amount of a Smo antagonist and a therapeutically effective amount of a BCR-ABL antagonist to a subject with chronic myelogenous leukemia; whereby the chronic myelogenous leukemia is treated.

Another aspect of the disclosure is a method of decreasing the number of hematopoietic malignancy stem cells in a tissue of a subject comprising administering a therapeutically effective amount of an Hh antagonist to a subject having a number of hematopoietic malignancy stem cells in a tissue; whereby the number of hematopoietic malignancy stem cells in the tissue of the subject is decreased.

Another aspect of the disclosure is a method of decreasing the number of chronic myelogenous leukemia stem cells in a tissue of a subject comprising administering a therapeutically effective amount of an Hh antagonist to a subject having a number of chronic myelogenous leukemia stem cells in a tissue; whereby the number of chronic myelogenous leukemia stem cells in the tissue of the subject is decreased.

Another aspect of the disclosure is a method of decreasing the number of chronic myelogenous leukemia stem cells in a tissue of a subject comprising administering a therapeutically effective amount a Smo antagonist to a subject having a number of chronic myelogenous leukemia stem cells in a tissue; whereby the number of chronic myelogenous leukemia stem cells in the tissue of the subject is decreased.

Another aspect of the disclosure is a method of decreasing the number hematopoietic malignancy stem cells in a tissue of a subject comprising administering a therapeutically effective amount of an Hh antagonist and a therapeutically effective amount of a BCR-ABL antagonist to a subject having a number of hematopoietic malignancy stem cells in a tissue; whereby the number of hematopoietic malignancy stem cells in the tissue of the subject is decreased.

Another aspect of the disclosure is a method of decreasing the number of chronic myelogenous leukemia stem cells in a tissue of a subject comprising administering a therapeutically effective amount of an Hh antagonist and a therapeutically effective amount of a BCR-ABL antagonist to a subject having a number of chronic myelogenous leukemia stem cells in a tissue; whereby the number of chronic myelogenous leukemia stem cells in the tissue of the subject is decreased.

Another aspect of the disclosure is a method of decreasing the number of chronic myelogenous leukemia stem cells in a tissue of a subject comprising administering a therapeutically effective amount of a Smo antagonist and a therapeutically effective amount of a BCR-ABL antagonist to a subject having a number of chronic myelogenous leukemia stem cells in a tissue; whereby the number of chronic myelogenous leukemia stem cells in the tissue of the subject is decreased.

### BRIEF DESCRIPTION OF THE DRAWINGS

FIG. 1 shows the conditional deletion of Smo impairs the development of BCR-ABL1 induced CML and depletes CML stem cells. Panel (a) of FIG. 1 shows real-time PCR results using primers specific for the loxP sites carried out on genomic DNA from Smo−/− mouse or Smo+/+ control mouse bone marrow (n=3, *P=0.03). RFU, relative fluorescent units. Panel (b) of FIG. 1 shows real-time PCR results using cDNAs of Smo transcripts from Smo−/− mouse or Smo+/+ control mouse c-Kit⁺Lin-^{/lo}Sca-1⁺Flk2⁻ (KLSF) cells (n=7, *P<0.00001). Panel (c) of FIG. 1 shows representative donor derived chimerism in lethally irradiated congenic recipient mice receiving a transplant of 500 KLSF cells from individual control Smo+/+ or Smo−/− mice at 20 weeks. Panel (d) of FIG. 1 shows a graph of the average donor derived chimerism in lethally irradiated congenic recipient mice receiving a transplant of 500 KLSF cells from individual control Smo+/+ or Smo−/− mice after long-term reconstitution (n=2 with 20 mice, P=0.0002). Each dot represents an individual mouse. Panel (e) of FIG. 1 shows the contribution to differentiated lineages from control Smo+/+ and Smo−/− cells following long-term bone marrow transplantation in peripheral blood (n=2 with 4 to 6 mice per cohort per experiment). Panel (f) of FIG. 1 shows a graph of average donor derived chimerism after long-term reconstitution following a secondary competitive repopulation analysis of control Smo+/+ mice and Smo knockout (Smo−/−) mice carried out using 1×10⁶ bone marrow cells from primary recipients isolated at 24 weeks and analyzed for donor chimerism in secondary recipients at 24 weeks (6 mice in each cohort p=0.04).

FIG. 2 shows the conditional deletion of Smo impairs the development of BCR-ABL induced CML and depletes CML stem cells. Panel (a) of FIG. 2 shows a survival curve for mice receiving 15,000 BCR-ABL infected control Smo+/+ or Smo−/− KLSF cells (n=3 with 34 mice, P=0.0002). Panel (b) of FIG. 2 shows a representative example of a flow cytometry analysis of CML stem cells (GFP⁺Lin-c⁻Kit⁺Sca⁺) in the bone marrow of mice transplanted with BCR-ABL1-infected control Smo+/+ or Smo−/− KLS cells. Panel (c) of FIG. 2 shows a graph of the average percentage of CML stem cells in mice receiving BCR-ABL1-infected control Smo+/+ or Smo−/− KLS cells (n=8 mice for Smo+/+ KLS cells and n=9 mice for Smo−/− KLS cells, *P=0.006).

FIG. 3 shows the presence of a constitutively active Smo protein increases the frequency of CML stem cells and accelerates CML disease. Panel (a) of FIG. 3 shows fluorescence activated cell sorting (FACS) analysis of YFP fluorescence, which reflects SmoM2 expression levels in control (con) and SmoM2 KLSF cells (n=4), in the KLSF population of bone marrow cells from control and SmoM2 mice. Panel (b) of FIG. 3 shows a FACS analysis of CML stem cells (GFP⁺ KLS) from mice receiving transplants of BCR-ABL1-infected control (left) and constitutively active Smo (SmoM2, right) bone marrow KLSF cells that is based on the detection of lin⁻GFP⁺ cells and c-Kit⁺Sca⁺ cells. Panel (c) of FIG. 3 shows a graph of the average percentage of CML stem cells (GFP⁺ KLS cells) in mice receiving transplants of BCR-ABL1-infected control and SmoM2 KLSF cells (n=4 for control and n=12 for SmoM2, P=0.048; error bars represent the standard error of the mean). Panel (d) of FIG. 3 shows the results of a real-time PCR analysis of SmoM2 transcription and expression in CML stem cells (GFP⁺ KLS). FIG. 4 shows the loss of Smo increases the frequency of cells with high levels of Numb protein and contributes to decreased CML growth. Panel (a) of FIG. 4 shows increased levels of Numb expression in CML stem cells from Smo−/− leukemia cells relative to control Smo+/+ cells when histological analyses are performed using a labeled Numb protein specific reagent and a DNA specific 4,6-diamidino-2-phenylindole (DAPI) stain. Panel (b) of FIG. 4 shows the average fluorescence intensity (AFI) of Numb staining in CML stem cells from Smo−/− leukemia cells relative to control Smo+/+ cells based on histological analyses of the type shown in panel (a) of FIG. 4. Panel (c) of FIG. 4 shows the frequency of cells with high expression levels of Numb (i.e., cells exhibiting mean fluorescence intensity values of greater than 1,000) based on a histological analyses of the type shown in panel (a) of FIG. 4 (n=3 using either CML KLS or CML c-Kit+ cells). Panel (d) of FIG. 4 shows that ectopic expression of the Numb protein inhibits long term serial replating of BCR-ABL1 transformed bone marrow KLSF cells. (Error bars show s.e.m. Note that from second replating, cells ectopically expressing Numb showed significantly reduced colony forming ability; 2^{nd} plating P=0.0067, 3^{rd} plating P=0.006 and 4^{th} plating P=0.002). Panel (e) of FIG. 4 shows decreased colony formation in methylcellulose media by CML KLS cells expressing Numb IRES-YFP relative to CML KLS cells receiving control vector (n=2; P=0.03).

FIG. 5 shows inhibition of Smo protein activity with cyclopamine treatment impairs CML development and progression. Panel (a) of FIG. 5 shows decreased colony formation in methycellulose media by KLS GFP+ CML stem cells treated with cyclopamine relative to control cells treated with tomatidine (n=2; P=0.005). Panel (b) of FIG. 5 shows a survival curve for cyclopamine treated mice and vehicle treated control mice receiving transplants of BCR-ABL1 infected KLS cells (n=3 with 32 mice, P=0.02). Panel (c) of FIG. 5 shows a representative flow cytometric analysis example of the frequency of CML stem cells in cyclopamine treated mice and vehicle treated control mice receiving transplants of BCR-ABL1 infected KLS cells. Panel (d) of FIG. 5 shows the average CML stem cell frequence in cyclopamine treated mice and vehicle treated control mice receiving transplants of BCR-ABL1 infected KLS cells (n=4, p=0.03). Panel (e) of FIG. 5 shows cyclopamine treatment of imatinib resistant bone marrow KLSF cells expressing the T3151 mutant BCR-ABL1 fusion protein and of imatinib sensitive bone marrow KLSF cells expressing BCR-ABL1 decreases colony formation in methycellulose media relative to untreated control cells (n=2; error bars show s.e.m).

FIG. 6 shows that deletion of Smo does not alter the frequency of hematopoietic lineages. Panel (a) of FIG. 6 shows the frequency of erythroid cells (Ter119), myeloid cells (Macl), B-cells (B220) or T-cells (CD4 and/or CD8) in whole bone marrow from Smo−/− mice or control Smo+/+ mice as assessed by FACS analysis (n=5). Panel (b) of FIG. 6 shows the frequency of KLSF cells in representative populations of whole bone marrow cells from Smo−/− mice or control Smo+/+ mice as assessed by FACS analysis. Panel (c) of FIG. 6 shows the average frequency of KLSF cells in representative populations of whole bone marrow cells from Smo−/− mice (n=10) or control Smo+/+ mice (n=10) as assessed by FACS analysis (error bars show s.e.m).

### DETAILED DESCRIPTION OF THE INVENTION

It will be appreciated that the following description is intended to provide details concerning specific representative aspects of the disclosure. It will also be appreciated that a wide variety of equivalents may be substituted for the specified elements of the methods described herein without departing from the spirit and scope of this disclosure as described in the appended claims. Additionally, all publications, including but not limited to patents and patent applications, cited in this disclosure are herein incorporated by reference as though fully set forth. Ranges identified herein are intended to include the values defining the upper and lower limits of a recited range, all discrete values within the range and any discrete sub-range within the range.

The term “hematopoietic cell malignancy” as used herein means malignancies of blood cells, or cells in tissues that produce blood cells or blood components. The blood cells may be red blood cells or white blood cells. The cells in tissues that produce blood cells or blood components may be cells in bone marrow or lymphatic tissue such as the lymph nodes. Hematopoietic cell malignancies may be cancers or other malignant conditions involving cells that form blood or blood Examples of hematopoietic cell malignancies that are cancers include leukemias, lymphomas and multiple myelomas. Examples of leukemias include acute lymphoblastic leukemia (ALL), acute myelogenous leukemia (AML) and chronic myelogenous leukemia (CML). Examples of lymphomas include Hodgkin's disease and its subtypes; non-Hodgkin lymphomas and its subtypes including chronic lymphocytic leukemia (CLL), small lymphocytic lymphoma (SLL), diffuse large B-cell lymphoma (DLBCL), follicular lymphoma (FL), mantle cell lymphoma (MCL), hairy cell leukemia (HCL), marginal zone lymphoma (MZL), Burkitt's lymphoma (BL), Post-transplant lymphoproliferative disorder (PTLD), T-cell prolymphocytic leukemia (T-PLL), B-cell prolymphocytic leukemia (B-PLL), Waldenström's macroglobulinemia/Lymphoplasmacytic lymphoma and other natural killer cell (NK-cell) or T-cell lymphomas. Examples of other malignant conditions which are hematopoietic cell malignancies include myelodysplastic syndrome (MDS); myeloproliferative diseases such as polycythemia vera (i.e., PV, PCV or polycythemia rubra vera (PRV)), essential thrombocytosis (ET), myelofibrosis; and diseases with features of both myelodysplastic syndromes and myeloproliferative diseases such as chronic myelomonocytic leukemia (CMML), juvenile myelomonocytic leukemia (JMML), atypical chronic myeloid leukemia (aCML) and myelodysplastic/myeloproliferative disease. Importantly, chronic myelogenous leukemia is one type of hematopoietic cell malignancy.

The term “chronic myelogenous leukemia” (CML) as used herein means a form of leukemia characterized by the uncontrolled proliferation of myeloid cells, or myelopoietic cells, in the bone marrow and the accumulation of these cells in the blood and other tissues. In CML large numbers of immature and mature granulocytic cell types are found in various tissues and in the blood. The total count of such cells may range from 1,000 to several hundred thousand per mm³. The predominant cell types are usually neutrophil, eosinophil or basophil granulocytes and even megakaryocytes. CML is a type of myeloproliferative disease associated with a characteristic chromosomal translocation called the Philadelphia chromosome which encodes oncogenic BCR-ABL proteins.

The term “subject” as used herein means an animal belonging to any genus for which treatment of a hematopoietic cell malignancy or chronic myelogenous leukemia is indicated, or decreasing the number of hematopoietic cell malignancy stem cells in a tissue of the animal or decreasing the number of chronic myelogenous leukemia stem cells in a tissue of the animal is indicated. One example of such a “subject” is a human such as a human patient.

The term “administering” means providing a compound to at least one tissue of a subject. Compounds may be administered to a subject corporeally or extra-corporeally. Extra-corporeal administration of a compound to a tissue of a subject occurs when a portion of a tissue, such as blood or bone marrow, is removed from the body of a subject, contacted with a compound that has been provided and a portion of the tissue contacted with the compounds is then returned to body of a subject.

The term “therapeutically effective amount” as used herein means those doses that, in a given individual subject, produce a response that results in improvement, or treatment, of one or more symptoms of a hematopoietic cell malignancy or chronic myelogenous leukemia (e.g., inflammatory cytokine levels) or results in a decrease in the number of hematopoietic malignancy stem cells or a decrease in the number of chronic myelogenous leukemia stem cells in a tissue of a subject. Therapeutically effective amounts, or doses, appropriate for an individual subject can be readily determined using routine clinical techniques well known by those of skill in the art (e.g., dose response plots).

The term “Hh antagonist” as used herein means a molecule that partially or completely inhibits, by any mechanism, an activity of a ligand or receptor necessary for signaling through the Hedgehog signal transduction path. An “Hh antagonist” may be a molecule that is capable of, directly or indirectly, substantially counteracting, reducing or inhibiting Hedgehog signal transduction pathway biological activity or Hedgehog signal transduction pathway activation. For example, an “Hh antagonist” may partially, or completely, inhibit the activity of Hedgehog signal transduction ligands such as the Sonic hedgehog (Shh) (e.g., SEQ ID NO: 1 or SEQ ID NO: 2), Indian hedgehog (Ihh) (e.g., SEQ ID NO: 3 or SEQ ID NO: 4) and Desert hedgehog (Dhh) (e.g., SEQ ID NO: 5 or SEQ ID NO: 6) ligands, or homologs of these, produced by cells. Alternatively, an “Hh antagonist” may block ligand binding to the Patched (Ptch) protein (e.g., SEQ ID NO: 7, SEQ ID NO: 8, SEQ ID NO: 9, SEQ ID NO: 10, SEQ ID NO: 11) or its homologs to prevent activation of a Smoothened protein (Smo) (e.g., SEQ ID NO: 12 or SEQ ID NO: 13) or homologs of the Smo protein. An “Hh antagonist” may also partially, or completely, inhibit the Smo seven-transmembrane receptor protein. Importantly, a “Smo antagonist” is one type of “Hh antagonist.”

The term “Smo antagonist” as used herein means a molecule that partially or completely inhibits, by any mechanism, an activity of a Smo receptor protein. A “Smo antagonist” may be a molecule that is capable of, directly or indirectly, substantially counteracting, reducing or inhibiting the biological activity of a Smo receptor protein or activation of a Smo receptor protein. A “Smo receptor protein” may include the Smoothened protein (SEQ ID NO: 12 or SEQ ID NO: 13) or homologs of this protein.

The term “BCR-ABL antagonist” as used herein means a molecule that partially or completely inhibits, by any mechanism, an activity of a BCR-ABL receptor tyrosine kinase. A “BCR-ABL antagonist” may be a molecule that is capable of, directly or indirectly, substantially counteracting, reducing or inhibiting the biological activity of a BCR-ABL receptor tyrosine kinase or activation of a BCR-ABL receptor tyrosine kinase. A “BCR-ABL receptor tyrosine kinase” may include a BCR-ABL fusion protein (e.g., SEQ ID NO: 15, SEQ ID NO: 16 or SEQ ID NO: 17) such as the p210 BCR-ABL1 fusion protein, or the P190 and P230 BCR-ABL fusion proteins or a homolog of these proteins.

The term “homolog” as used herein means protein sequences having between 85% and 100% sequence identity to a reference sequence. For example, homologs of the Homo sapiens Shh protein shown in SEQ ID NO: 2 would include those peptide chains that have between 85% and 100% sequence identity to SEQ ID NO: 2, homologs of the Homo sapiens Ihh protein shown in SEQ ID NO: 4 would include those peptide chains that have between 90% and 100% sequence identity to SEQ ID NO: 4, homologs of the Homo sapiens Dhh protein shown in SEQ ID NO: 6 would include those peptide chains that have between 85% and 100% sequence identity to SEQ ID NO: 6, homologs of the Homo sapiens Ptch protein shown in SEQ ID NO: 8 would include those peptide chains that have between 90% and 100% sequence identity to SEQ ID NO: 8; homologs of the Homo sapiens Ptch protein shown in SEQ ID NO: 9 would include those peptide chains that have between 90% and 100% sequence identity to SEQ ID NO: 9, homologs of the Homo sapiens Ptch protein shown in SEQ ID NO: 10 would include those peptide chains that have between 90% and 100% sequence identity to SEQ ID NO: 10, homologs of the Homo sapiens Ptch protein shown in SEQ ID NO: 11 would include those peptide chains that have between 90% and 100% sequence identity to SEQ ID NO: 11, homologs of the Homo sapiens Smo protein shown in SEQ ID NO: 13 would include those peptide chains that have between 90% and 100% sequence identity to SEQ ID NO: 13, homologs of the Homo sapiens BCR-ABL protein shown in SEQ ID NO: 16 would include those peptide chains that have between 85% and 100% sequence identity to SEQ ID NO: 16, and homologs of the Homo sapiens BCR-ABL protein shown in SEQ ID NO: 17 would include those peptide chains that have between 85% and 100% sequence identity to SEQ ID NO: 17. Percent identity between two peptide chains can be determined by pair wise alignment using the default settings of the AlignX module of Vector NTI v.9.0.0 (Invitrogen Corp., Carslbad, Calif.).

The term “peptide chain” means a molecule that comprises at least two amino acid residues linked by a peptide bond to form a chain. Large peptide chains of more than 50 amino acids may be referred to as “polypeptides” or “proteins.” Small peptide chains of less than 50 amino acids may be referred to as “peptides.”

“Antagonist” compounds useful in the methods of the disclosure may comprise, for example, small organic molecules, peptide chains, antibodies, antibody fragments, polynucleotides or combinations of these. Such antagonists may, for example, disrupt the activity of the Hh pathway, Smo proteins or BCR-ABL proteins by preventing activation or formation of functional complexes comprising Hh pathway components (e.g., ligands and receptors) or Smo proteins and BCR-ABL proteins.

Antagonists useful in the methods of the disclosure may also be nucleic acid molecules. Such nucleic acid molecules may be interfering nucleic acid molecules such as short interfering RNAs or antisense molecules that are antagonists of the activity of an Hh pathway component such as the Smo protein. Alternatively, polynucleotide molecules such as double and single stranded plasmid DNA vectors, artificial chromosomes, or linear nucleic acids or other vectors that encode an antagonist (e.g., peptide chain or RNA), or function as an antagonist, may be used in the methods of the disclosure to administer an antagonist to a subject.

The term “hematopoietic malignancy stem cells” as used herein refers to cells with the ability to give rise to all cell types found in a particular hematopoietic cell malignancy. Hematopoietic malignancy stem cells generate hematopoietic cell malignancies through the stem cell processes of self-renewal and differentiation into multiple hematopoietic malignancy cell types. Hematopoietic malignancy stem cells may persist in a tissue of a subject as a distinct population and can cause relapse and metastasis by giving rise to new hematopoietic cell malignancies. Hematopoietic malignancy stem cells are able to produce hematopoietic cell malignancy when they are transferred to a new host such as an immune compromised animal (e.g., NOD mice, SCID mice or irradiated mice). Hematopoietic malignancy stem cells in acute myelogenous leukemia may have a CD34⁴1CD38⁻ phenotype. Hematopoietic malignancy stem cells in chronic myelogenous leukemia, such as chronic myelgenous leukemia stem cells, may also have a c-Kit⁺Lin⁻Sca-1⁺phenotype. See e.g., Hu et al., 103 Proc. Natl. Acad. Sci. USA 16870 (2006). Alternatively, hematopoietic malignancy stem cells in chronic myelogenous leukemia, such as chronic myelgenous leukemia stem cells, may have a c-Kit⁺Lin^{-/lo}Sca-1⁺Flk2⁻ phenotype. Chronic myelogenous leukemia stem cells are one type of hematopoietic malignancy stem cell.

The term “chronic myelogenous leukemia stem cells” (CML stem cells) as used herein refers to cells with the ability to give rise to all cell types found in chronic myelogenous leukemia. Chronic myelogenous leukemia stem cells generate chronic myelogenous leukemia through the stem cell processes of self-renewal and differentiation into the multiple cell types associated with chronic myelogenous leukemia. Chronic myelogenous leukemia stem cells may persist in a tissue of a subject as a distinct population and can cause relapse and metastasis by giving rise to new chronic myelogenous leukemias such as drug resistant leukemias (e.g., imatinib resistant chronic myelogenous leukemia). Chronic myelogenous leukemia stem cells are able to produce chronic myelogenous leukemia malignancy when they are transferred to a new host such as an immune compromised animal (e.g., SCID mice or irradiated mice) and can be functionally identified on this basis. Chronic myelogenous leukemia stem cells may also have a c-Kit⁺Lin⁻Sca-1⁺ phenotype. See e.g., Hu et al., 103 Proc. Natl. Acad. Sci. USA 16870 (2006). Alternatively, chronic myelogenous leukemia stem cells may have a c-Kit⁺Lin^{-/lo}Sca-1⁺Flk2⁻ phenotype.

The term “tissue” as used herein means a collection of similar cells and the intercellular substances surrounding them. Examples of tissues include epithelium, connective tissues including adipose tissue, blood, bone, bone marrow, cartilage, muscle tissue and nerve tissue.

One aspect of the disclosure is a method of treating a hematopoietic cell malignancy in a subject comprising administering a therapeutically effective amount of an Hh antagonist to a subject with a hematopoietic cell malignancy; whereby the hematopoietic cell malignancy is treated.

Examples of Hh antagonists include steroidal alkaloids, such as cyclopamine, and derivatives thereof, and other small molecules such as SANT-1, SANT-2, SANT-3 and SANT-4 that can reduce or inhibit Hh pathway activity by directly repressing Smo protein activity. Derivatives of cyclopamine, including cyclopamine salts, may be Hh antagonists and are also Smo antagonists. Cyclopamine has the structure:

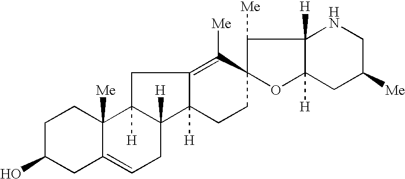

Hh antagonist may include derivatives of a given antagonist. Cyclopamine salts, such as cyclopamine tartarate salts, are one example of such derivatives and are well known in the art. Cyclopamine derivatives, such as cyclopamine tartarate salts and other cyclopamine derivatives, are disclosed in U.S. Pat. No. 7,230,004 and US 2007/0191410 A1 which are both entitled “Cyclopamine analogues and methods of use thereof[.]” In addition, cholesterol can be required for Hh pathway activity and agents that reduce the availability of cholesterol, for example, by removing it from cell membranes, can act as Hh pathway antagonists. See e.g., Cooper et al., 33 Nat. Genet. 408 (2003); see also Cooper et al., 34 Nat. Genet. 113 (2003).

Other examples of Hh antagonists include antibodies which bind to one or more ligands such as Shh, Ihh or Dhh and decrease ligand stimulated Hh pathway activity or bind to a Ptch receptor and decrease ligand stimulated Hh pathway activity (e.g., by antagonizing a ligand binding site on a Ptch receptor). Such Hh antagonist antibodies may also bind homologs of Hh pathway ligands or receptors.

Examples of Hh antagonists for use in the methods of the disclosure also include itraconazole, salinomycin sodium, oligomycin, colchicine, podophyllum resin, croton oil, ipecac syrup, vindesine, vincristine sulfate, demecolcine, vinorelbine tartrate, loxapine succinate, cyproheptadine, itraconazole, colchiceine, pimethixene maeate, diaziquone, sulfisomidine, cyclohexamide, cyclopamine, cod liver oil, methoxyvone, promethazine hydrochloride, sulfaquinoxaline sodium, vinblastine sulfate, hydroxyzine, eucalyptol, rotenone, phenoxybenzamine hydrochloride, 5-azacytidine, W-7 hydrochloride, dihydroartemisinin, clompramine, raloxifine hydrochloride, doxazosin mesylate salt, dihydroartemisinin, comipramine, raloxafine hydrochloride, doxazpsin mesylate salt, chloroquine diphosphate salt, imipramine, thioridiazine, clothiapine, zolantidine, crassin acetate, estriol benzyl ether, fluphenazine N-mustard (SKF-7171A), almond oil, promazine hydrochloride, estradiol acetate, trimipramine maleate, copper (II) acetate, estradiol 3-benzoate, Amitriptyline, Chlorquinaldol (5,7-Dichloro-2-methyl-8-quinolinol) and derivatives of these.

The mode of administration of the Hh antagonists such as Smo antagonists or BCR-ABL antagonists in the methods of the disclosure may be any suitable route that delivers these antagonists to a subject. The small organic molecules, peptide chains, antibodies, antibody fragments, polynucleotides or combinations of these and pharmaceutical compositions comprising these agents are particularly useful for parenteral administration, i.e., intrarticularly, subcutaneously, intramuscularly, intradermally, intravenously or intranasally.

Antagonists useful in the methods of the disclosure may be prepared as pharmaceutical compositions containing an effective amount of the antagonist as an active ingredient in a pharmaceutically acceptable carrier. An aqueous suspension or solution containing the antagonist, preferably buffered at physiological pH, in a form ready for injection is preferred. The compositions for parenteral administration will commonly comprise a solution of the antagonist or a cocktail thereof dissolved in an pharmaceutically acceptable carrier, preferably an aqueous carrier. A variety of aqueous carriers may be employed, e.g., 0.4% saline, 0.3% glycine and the like. These solutions are sterile and generally free of particulate matter. These solutions may be sterilized by conventional, well-known sterilization techniques (e.g., filtration). The compositions may contain pharmaceutically acceptable auxiliary substances as required to approximate physiological conditions such as pH adjusting and buffering agents, etc. The concentration of the antagonist in such pharmaceutical formulations can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition useful in the methods of the disclosure for injection intrarticularly could be prepared to contain 1 mL sterile buffered water, and between about 1 ng to about 100 mg, e.g., about 50 ng to about 30 mg or more preferably, about 5 mg to about 25 mg, of an antagonist. Similarly, a pharmaceutical composition for intravenous injection could be made up to contain about 250 ml of sterile Ringer's solution, and about 1 mg to about 30 mg and preferably 5 mg to about 25 mg of an antagonist. Actual methods for preparing parenterally administrable compositions are well known and are described in more detail in, for example, “Remington's Pharmaceutical Science,” 15th ed., Mack Publishing Company, Easton, Pa. Doses of antagonists may be between about 0.01 mg per kg of subject body weight or 35 mg per kg of subject body weight.

The antagonists useful in the methods of the disclosure, when in a pharmaceutical preparation, can be administered in unit dose forms. The appropriate therapeutically effective amount, or dose, can be determined readily by those of skill in the art. A determined dose may, if necessary, be repeated at appropriate time intervals selected as appropriate by a physician during the period of administration.

The antagonists useful in the methods of the disclosure can be lyophilized for storage and reconstituted in a suitable carrier prior to use. This technique has been shown to be effective with conventional immunoglobulins and protein preparations and art-known lyophilization and reconstitution techniques can be employed.

In some embodiments of the methods of the disclosure the antagonist is an isolated antibody reactive with an Hh pathway component such as a Smo protein or an BCR-ABL protein. An antibody is reactive with a protein when, for example, it specifically binds a given peptide chain (e.g., Homo sapiens Ptch protein) or a complex comprising a given peptide chain. The binding of an antagonist, such as an antibody reactive with a given peptide chain, is specific for the given peptide chain when such binding can be used to detect the presence of the given peptide chain (e.g., Homo sapiens Ptch protein), but not a second non-homologous peptide chain (e.g., albumin). This specific binding can be used to distinguish the two peptide chains from each other. Specific binding can be assayed using conventional techniques such as ELISAs and Western blots as well as other techniques well known in the art.

Exemplary antibody antagonists may be antibodies of the IgG, IgD, IgGa or IgM isotypes. Additionally, such antagonist antibodies can be post-translationally modified by processes such as glycosylation, isomerization, aglycosylation or non-naturally occurring covalent modification such as the addition of polyethylene glycol moieties (pegylation) and lipidation. Such modifications may occur in vivo or in vitro. Fully human, humanized and affinity-matured antibody molecules or antibody fragments are useful in the methods of the disclosure as are fusion proteins and chimeric proteins comprising antibody fragments.

The antibody antagonists useful in the methods of the disclosure may specifically bind a given protein or complexes comprising a given protein with a K_{d} less than or equal to about 10⁻⁷, 10⁻⁸, 10⁻⁹, 10⁻¹⁰, 10⁻¹¹ or 10⁻¹² M. The affinity of an antibody agonist molecule for a given ligand, receptor or complex comprising these can be determined experimentally using any suitable method. Such methods may utilize BIACORE™ or KINEXA™ instrumentation, ELISA or competitive binding assays known to those skilled in the art.

Antibody and peptide chain antagonist molecules binding a given protein homolog with a desired affinity can be selected from libraries of protein variants or fragments by techniques including antibody affinity maturation and other art-recognized techniques suitable for non-antibody molecules.

Another example of an Hh antagonist are arsenical agents including arsenic trioxide (As₂O₃) and sodium arsenite (NaAsO₂).

In one embodiment of the method of the disclosure the Hh antagonist is selected from the group consisting of cyclopamine, cyclopamine salts and arsenic trioxide.

Another aspect of the disclosure is a method of treating chronic myelogenous leukemia in a subject comprising administering a therapeutically effective amount of an Hh antagonist to a subject with chronic myelogenous leukemia; whereby the chronic myelogenous leukemia is treated.

Another aspect of the disclosure is a method of treating chronic myelogenous leukemia in a subject comprising administering a therapeutically effective amount of a Smo antagonist to a subject with chronic myelogenous leukemia; whereby the chronic myelogenous leukemia is treated.

In another embodiment of the method of the disclosure the Smo antagonist is selected from the group consisting of cyclopamine and cyclopamine salts.

In another embodiment of the method of the disclosure the Smo antagonist is cyclopamine.

Another aspect of the disclosure is a method of treating a hematopoietic cell malignancy in a subject comprising administering a therapeutically effective amount of an Hh antagonist and a therapeutically effective amount of a BCR-ABL antagonist to a subject with a hematopoietic cell malignancy; whereby the hematopoietic cell malignancy is treated.

In another embodiment of the method of the disclosure the BCR-ABL antagonist is selected from the group consisting of imantinib, imatinib mesilate, dasatinib and nilotinib.

Imantinib has the structure shown below:

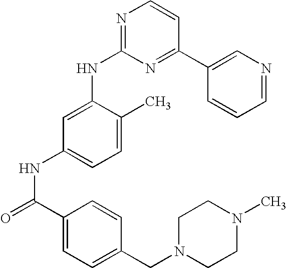

Imatinib mesilate has the structure shown below:

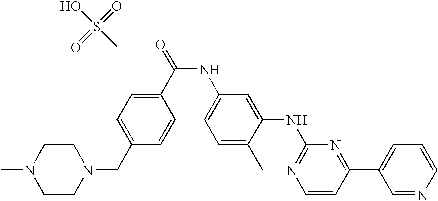

Dasatinib has the structure shown below:

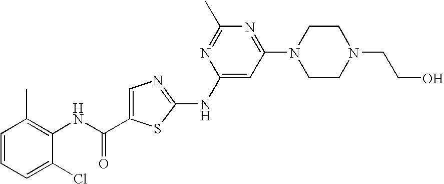

Nilotinib has the structure shown below:

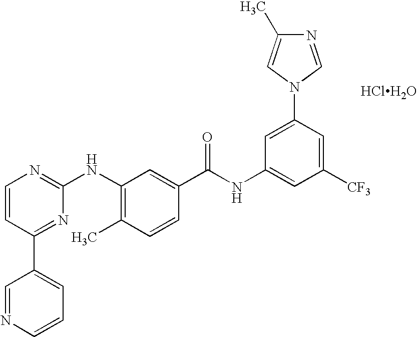

Another aspect of the disclosure is a method of treating chronic myelogenous leukemia in a subject comprising administering a therapeutically effective amount of an Hh antagonist and a therapeutically effective amount of a BCR-ABL antagonist to a subject with chronic myelogenous leukemia; whereby the chronic myelogenous leukemia is treated.

In another embodiment of the method of the disclosure the Hh antagonist is selected from the group consisting of cyclopamine, cyclopamine salts and arsenic trioxide; and the BCR-ABL antagonist is selected from the group consisting of imantinib, imatinib mesilate, dasatinib and nilotinib.

Another aspect of the disclosure is a method of treating chronic myelogenous leukemia in a subject comprising administering a therapeutically effective amount of a Smo antagonist and a therapeutically effective amount of a BCR-ABL antagonist to a subject with chronic myelogenous leukemia; whereby the chronic myelogenous leukemia is treated.

In another embodiment of the method of the disclosure the Smo antagonist is selected from the group consisting of cyclopamine and cyclopamine salts; and the BCR-ABL antagonist is selected from the group consisting of imantinib, imatinib mesilate, dasatinib and nilotinib.

In another embodiment of the method of the disclosure the Smo antagonist is cyclopamine and the BCR-ABL antagonist is imantinib.

Another aspect of the disclosure is a method of decreasing the number of hematopoietic malignancy stem cells in a tissue of a subject comprising administering a therapeutically effective amount of an Hh antagonist to a subject having a number of hematopoietic malignancy stem cells in a tissue; whereby the number of hematopoietic malignancy stem cells in the tissue of the subject is decreased. The number of hematopoietic malignancy stem cells in a tissue can be readily determined using a variety of different methods well known in the art. Typically, a tissue sample, such as blood or bone marrow, is first obtained from a subject. Importantly, some preparation of tissue samples such as maceration and trypsinization may be necessary to facilitate a determination of the number of hematopoietic malignancy stem cells present in a tissue. However, for some tissues, such as blood, this may not be necessary. Next, the number of cells exhibiting a hematopoietic malignancy stem cell phenotype in a tissue of a subject is determined. This is done by using a hemocytometer, flow cyctometric analyses such as FACS analysis, or any other assay, such as growth assays (e.g., growth in carboxymethycellulose media), appropriate for identifying cells exhibiting a particular hematopoietic malignancy stem cell phenotype (e.g., hematopoietic malignancy stem cells with a CD34⁺/CD38⁻ phenotype in acute myelogenous leukemia and hematopoietic malignancy stem cells with a c-Kit⁺Lin⁻Sca-1⁺ phenotype or a c-Kit⁺Lin^{-/lo}Sca-1⁺Flk2⁻ in chronic myelogenous leukemia). Hematopoietic malignancy stem cells numbers can be expressed in any manner appropriate for a particular assay such as number of cells per a unit of tissue sample volume (e.g., mm³). Moreover, to confirm there is a decrease in hematopoietic malignancy stem cells, such as chronic myelogenous leukemia stem cells, in a tissue of a subject it may be necessary to count the number of such cells initially present in a tissue of a subject prior to performing the methods of the disclosure and again after the methods of the disclosure have been performed.

Another aspect of the disclosure is a method of decreasing the number of chronic myelogenous leukemia stem cells in a tissue of a subject comprising administering a therapeutically effective amount of an Hh antagonist to a subject having a number of chronic myelogenous leukemia stem cells in a tissue; whereby the number of chronic myelogenous leukemia stem cells in the tissue of the subject is decreased.

Another aspect of the disclosure is a method of decreasing the number of chronic myelogenous leukemia stem cells in a tissue of a subject comprising administering a therapeutically effective amount of a Smo antagonist to a subject having a number of chronic myelogenous leukemia stem cells in a tissue; whereby the number of chronic myelogenous leukemia stem cells in the tissue of the subject is decreased.

Another aspect of the disclosure is a method of decreasing the number hematopoietic malignancy stem cells in a tissue of a subject comprising administering a therapeutically effective amount of an Hh antagonist and a therapeutically effective amount of a BCR-ABL antagonist to a subject having a number of hematopoietic malignancy stem cells in a tissue; whereby the number of hematopoietic malignancy stem cells in the tissue of the subject is decreased.

Another aspect of the disclosure is a method of decreasing the number of chronic myelogenous leukemia stem cells in a tissue of a subject comprising administering a therapeutically effective amount of an Hh antagonist and a therapeutically effective amount of a BCR-ABL antagonist to a subject having a number of chronic myelogenous leukemia stem cells in a tissue; whereby the number of chronic myelogenous leukemia stem cells in the tissue of the subject is decreased.

Another aspect of the disclosure is a method of decreasing the number of chronic myelogenous leukemia stem cells in a tissue of a subject comprising administering a therapeutically effective amount of a Smo antagonist and a therapeutically effective amount of an BCR-ABL antagonist to a subject having a number of chronic myelogenous leukemia stem cells in a tissue; whereby the number of chronic myelogenous leukemia stem cells in the tissue of the subject is decreased.

### EXAMPLES

### Materials and Experimental Methods

Mice: The Smoothened conditional knockout mice used were in a mixed 129X1/SvJ and C57BL/6J background. Transplant recipients (C57BL/Ka CD45.1) were 8-10 weeks of age. All mice were bred and maintained on acidified, antibiotic water in an animal care facility. All animal experiments were performed according to approved animal care and use protocols.

Hematopoietic Stem Cell (HSC) Isolation and Analysis: Isolation of HSCs from bone marrow and their transplantation for in vivo analysis of function were performed as described Long et al. See Long et al., 128 Development 5099 (2001). For analysis of lineage markers, bone marrow cells from control or Smo^{-/-} mice were incubated with antibodies to murine Ter119, Mac-1, B220, and CD3 (eBiosciences Inc., San Diego, Calif.) and analyzed by FACS. For reconstitution assays, bone marrow cells from control, Smo−/− or SmoM2 mice were stained and analyzed for the c-Kit⁺Lin^{-/lo}Sca-1⁺Flk2⁻ (KLSF) cells¹⁹ and 500 control or Smo−/− KLSF cells were injected along with 200,000 competing bone marrow cells into lethally irradiated CD45.1 recipients. Multilineage repopulation was assessed at 20 weeks. Secondary repopulation assays were carried out by isolating whole bone marrow from primary recipients originally transplanted with control or Smo−/− KLSF cells. Recipient mice were sacrificed and analyzed for donor chimerism at 24 weeks.

Generation and Analysis of Diseased Mice: HSCs from Smo−/− or control mice were isolated and cultured overnight in X-VIVO™ media (Cambrex Bio Science Walkersville Inc., Walkersville, Md.) with 10% FBS, 100 ng/ml SCF, and 20 ng/ml TPO in a 96 well U-bottom plate (50,000 per well). Subsequently, cells were infected with MSCV-BCR-ABL-IRES-GFP or MSCV-IRES-GFP as a control. Cells were harvested 48 hours later and transplanted retro-orbitally with 200,000 whole bone marrow cells into lethally irradiated allelically mismatched recipients. After transplantation, recipient mice were evaluated daily for signs of morbidity, weight loss, failure to groom and splenomegaly. Premorbid animals were sacrificed and relevant tissues were harvested and analyzed by flow cytometry and histopathology. For flow cytometric analysis of CML stem cells, leukemic cell were stained for KLS and analyzed on FACS-VANTAGE™ (BD Biosciences Inc., San Jose, Calif.) and FLOWJO™ software (Tree Star Inc., Ashland, Oreg.).

Cyclopamine Treatment of Diseased Mice: Mice were given 25 mg/kg of cyclopamine twice a day by oral gavage beginning 6-8 days after transplantation. Treatment was continued for five consecutive days, stopped for two days, then continued once a day till the end of the monitoring period. Mice were visually assessed daily for signs of distress. In mice that displayed signs of distress treatment was stopped and continued when they showed recovery.

Methylcellulose Colony Formation Assays: For the colony formation assays CML KLSGFP⁺ cells, or KLSF cells infected with wild type BCR-ABL or T315I BCR-ABL were sorted into a 24 well plate with complete M3434 methylcellulose medium (Stem Cell Technologies Inc., Vancouver, BC, Canada) containing cyclopamine (Toronto Research Chemicals Inc., North York, ON, Canada) and/or imatinib (Sequoia Research Chemicals Ltd., Pangbourne, United Kingdom). Colonies were counted 8-10 days after plating. For the serial replating assay, cells were harvested and counted and 10,000 cells were replated into 12-well plates.

Immunofluorescence Staining: Primary CML cells harvested from bone marrow and spleen were sorted for KLS and GFP⁺ cells by FACS. Cells were cytospun, airdried and fixed in 4% paraformaldehyde. The primary antibody used was a NB 100-874 goat anti-Numb polyclonal antibody preparation (Novus Biologicals Inc., Littleton, Colo.) and the secondary antibody used was A11058 donkey anti-goat-ALEXA FLUOR® 594 polyclonal antibody preparation (Invitrogen Corp., Carlsbad, Calif.). DAPI (Invitrogen Corp., Carlsbad, Calif.) was added as a nuclear counter-stain. Slides were viewed on the AXIO™ Imager (Carl Zeiss IMT Corp., Maple Grove, Minn.) at 630× magnification. Fluorescence intensity analysis was quantified using METAMORPH™ software (MDS Inc., Downingtown, Pa.).

Real-Time RT-PCR Analysis: RNA was isolated using RNAQUEOUS™-Micro (Ambion) and converted to cDNA using SUPERSCRIPT™ II (Invitrogen Corp., Carlsbad, Calif.). cDNA concentrations were measured with a fluorometer (Turner Designs Inc., Sunnyvale, Calif.) using RIBOGREEN® reagent (Invitrogen Corp., Carlsbad, Calif.). Quantitative real-time PCR was performed using an ICYCLER™ (BioRad Laboratories Inc., Hercules, Calif.) by mixing equal amounts of cDNAs, IQ™ SYBR® Green Supermix (BioRad Laboratories Inc., Hercules, Calif.) and gene specific primers. All real time data was normalized to β-actin mRNA transcript levels.

### Example 1

The Smoothened (Smo) gene was conditionally deleted in mice. As discussed above, the Smo protein encoded by this gene is a seven-transmembrane protein with an essential role in the Hh signal transduction pathway. Smo is normally negatively regulated by the Hh receptor, Patched (Ptch), in the absence of ligand. This inhibition is relieved when Ptch is bound by any of the three mammalian Hh proteins, Shh, Ihh or Dhh. Subsequently, Smo activation causes transcriptional activation of Hh pathway targets via the Gli family of transcriptional effectors.

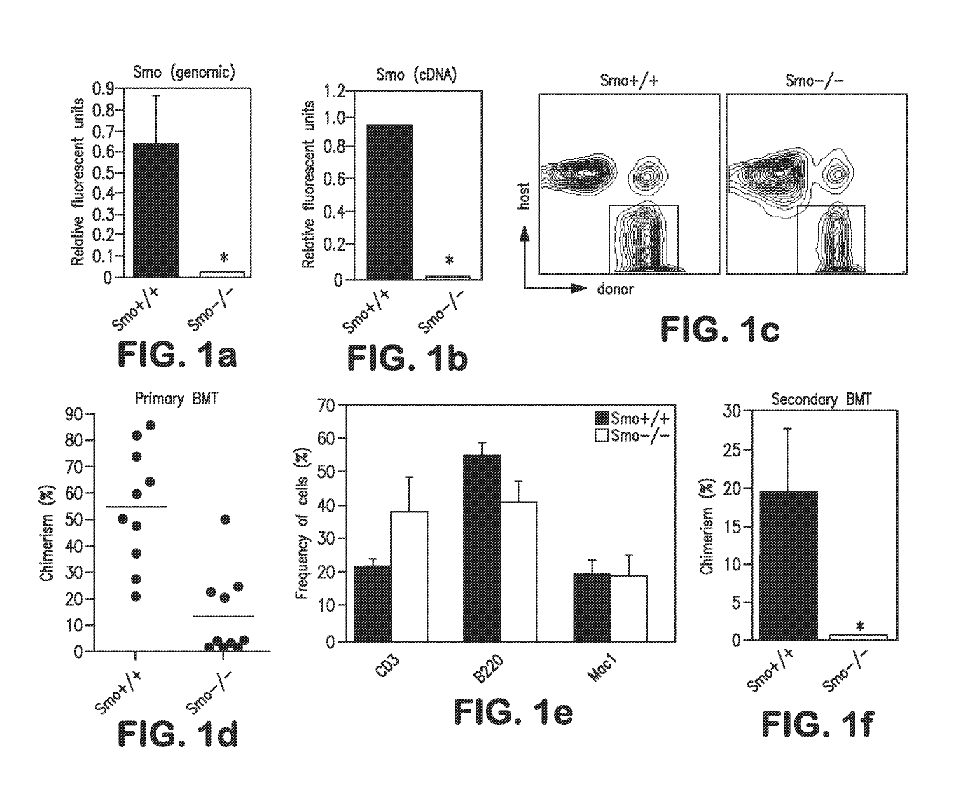

To conditionally delete Smo function in the hematopoietic system, we crossed mice carrying a Smo allele flanked by loxP sites to mice in which Cre recombinase expression is driven by Vav regulatory elements. Vav-Cre transgenics are effective for deleting genes of interest in hematopoietic stem cells (HSCs) as well as all other hematopoietic cells beginning in embryonic development. To examine the efficiency of Smo deletion, we isolated bone marrow cells from either Smo^{loxP/loxP}; Vav-Cre/+ mice or Smo^{loxP/loxP}; +/+ control mice and assayed for the presence of the region flanked by loxP by real time PCR analysis. The presence of Vav-Cre efficiently deleted Smo in these cells (FIG. 1, panel (a)). We also examined changes in the expression of Smo mRNA by real time RT-PCR analysis in HSCs and found that it was undetectable in the HSC enriched c-Kit⁺Lin-^{/lo}Sca-1⁺Flk2⁻ (KLSF) population from Smo^{loxP/loxP}; Vav-Cre/+ mice (FIG. 1, panel b). These data show that Smo can be efficiently deleted in the hematopoietic system and in HSCs and that the Smo^{loxP/loxP}; Vav-Cre/+ mouse (henceforth referred to as Smo−/−) is a valuable model for testing the role of Hh signaling in hematopoietic development and leukemia progression in vivo.

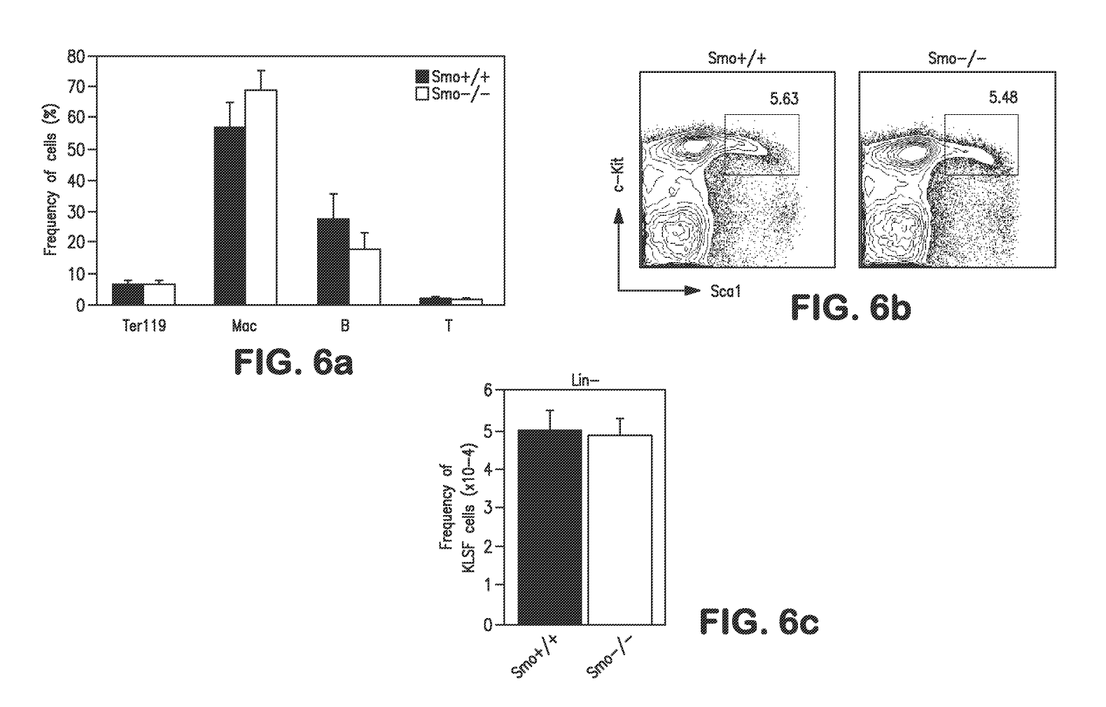

We tested whether loss of Smo leads to alterations in the development or function of HSCs. Bone marrow analysis of adult Smo−/− mice revealed no differences in the frequency of differentiated cells or in the frequency of phenotypic HSCs (FIG. 6). Loss of Smo, however, led to a clear defect in long-term HSC function. Transplantation of KFLS cells into lethally irradiated mice thus revealed a 3.6 fold reduction in donor chimerism in mice reconstituted with Smo−/− as compared to wild type HSCs (FIG. 1, panel (c); FIG. 1, panel (d)), although the relative contribution of Smo−/− KLSF cells to differentiated lineages was similar to wild-type (FIG. 1, panel (e)). In striking contrast to wild-type cells, the level of reconstitution by Smo−/− cells upon secondary transplantation was nearly undetectable (FIG. 1, panel (f)). Consistent with this requirement for Smo, HSCs expressed Gli1 and Ptch pathway components that can act as targets of Hh signaling (data not shown). Cumulatively, these data show that Smo is active in HSCs and required for their long term renewal in vivo.

Additional details concerning the experiment results shown in FIG. 1 are as described here. FIG. 1 shows conditional deletion of Smoothened impairs long term hematopoietic stem cell transplantation ability. FIG. 1, panel (a) shows confirmation of genomic deletion in Smo−/− mice. Genomic DNA was synthesized from whole bone marrow from Smo−/− or control mice, and real time PCR carried out using primers specific for the region flanked by the lox-P sites. Data shown is an average of three independent experiments. P=0.03. FIG. 1, panel (b) shows confirmation of reduction of Smo expression in Smo−/− mice. cDNA was synthesized from sorted KLSF cells from Smo−/− or control Smo+/+ mice, and real time PCR carried out using primers specific for Smo. Data shown is an average of seven independent experiments, p<0.00001. For FIG. 1, panel (c) and FIG. 1, panel (d) 500 KLSF cells from control Smo+/+ or Smo−/− mice were transplanted together with competing bone marrow cells into lethally irradiated congenic recipients and donor derived chimerism monitored for 16-24 weeks. FIG. 1, panel (c) shows representative plots of donor derived chimerism from individual mice at 20 weeks, Smo+/+, left and Smo−/− right. FIG. 1, panel (d) shows a graph of average donor derived chimerism after long-term reconstitution (4-6 mice in each cohort for a total of twenty mice over two independent experiments, P=0.0002). Each dot represents an individual mouse. FIG. 1, panel (e) shows the contribution to differentiated cell lineages from control Smo+/+ and Smo−/− cells following long-term bone marrow transplantation in peripheral blood. Results are representative of two independent experiments with 4-6 mice per cohort per experiment. FIG. 1, panel (f) shows the results of a secondary competitive repopulation analysis of control (Smo+/+) and Smoothened knockout (Smo−/−) mice carried out using 1×10⁶ bone marrow cells from primary recipients isolated at 24 weeks and analyzed for donor chimerism in secondary recipients at 24 weeks. FIG. 1, panel (f) is a graph of average donor derived chimerism after long-term reconstitution (6 mice in each cohort p=0.04).

Additional details concerning the experiment results shown in FIG. 6 are as described here. FIG. 6 shows that deletion of Smo does not alter the frequence of hematopoietic lineages. In FIG. 6, panel (a) bone marrow cells from control or Smo −/− mice were analyzed for frequency of cells of distinct hematopoietic lineages. Whole bone marrow cells were stained with erythroid (Ter119), myeloid (Macl), B (B220) or T (CD4 and/or CD8) markers and analyzed by FACS. Data shown is an average of results from five mice. In FIG. 6, panel (b) bone marrow cells from control or Smo−/−mice were analyzed for frequency of KLSF cells. Dot plots are shown for one representative control Smo+/+ (left) and Smo−/− (right). FIG. 6, panel (c) shows the average frequency of KLSF cells in control and Smo−/− mice (n=10). Error bars show s.e.m.

### Example 2

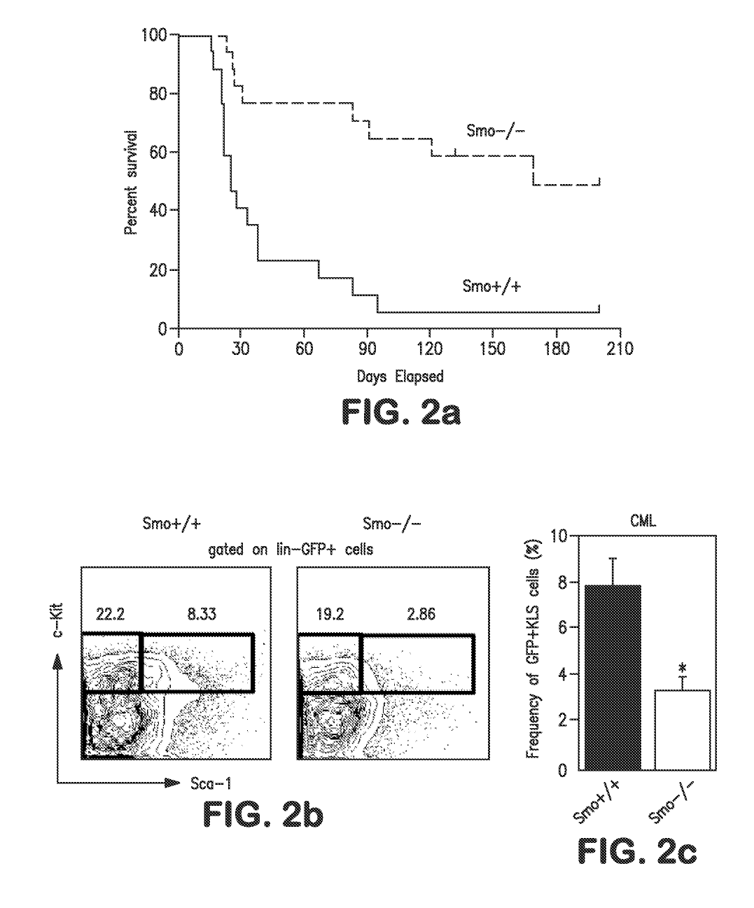

We investigated the role of Hh signaling in CML initiation and progression. CML can be effectively modeled in mice by transducing the p210 form of BCR-ABL1 into hematopoietic progenitors and transplanting these cells into lethally irradiated mice. To test the role of Hh signaling in this mouse model of CML, HSCs from control or Smo−/− mice were infected with viruses carrying BCR-ABL1 and transplanted after infection. Transduction of BCR-ABL1 into control cells resulted in CML in 94% of mice (16/17; FIG. 2, panel (a)) within 3 months. In contrast, only 47% of the mice transplanted with similarly transduced Smo−/− cells succumbed to CML (8/17; FIG. 2, panel (a)) despite being monitored for nearly 7 months. Furthermore the mice that succumbed to leukemia from Smo−/− cells exhibited an increased latency of the disease. These data show that Hh activity is required for the normal initiation and progression of CML.

The propagation of several cancers has been shown to depend on cancer stem cells, a critical subset of cancer cells capable of transferring cancer to a new host. The cancer stem cell that drives CML has been previously identified as c-kit⁺LinSca-1⁺ cells (KLS). Given our observation that the lack of Smo function led to impaired growth of CML, we examined the effect of Smo loss on CML stem cells and found, remarkably, that the frequency of CML stem cells was significantly reduced in the absence of Smo (FIG. 2, panel (b); FIG. 2, panel (c)). As these CML stem cells (KLS cells) have been shown to be responsible for propagating CML, their loss in the Smo−/− background would explain both the delayed progression and the reduced incidence of leukemia we observed.

Additional details concerning the experiment results shown in FIG. 2 are as described here. FIG. 2 show that conditional deletion of Smoothened impairs the development of BCR-ABL induced chronic myelogenous leukemia and depletes CML stem cells. FIG. 2, panel (a) shows a survival curve for mice receiving 15,000 BCR-ABL infected control Smo+/+ or Smo−/− KLSF cells. Cumulative data is shown from three independent experiments, with a total of 34 mice (p=0.0002). Similar results were obtained from another independent experiment with 8 mice for Smo+/+ and 10 mice for Smo−/− using BCR-ABL transduced 5-FU (150 mg/kg) treated whole bone marrow cells. FIG. 2, panel (b) shows a representative example of bone marrow cells from mice transplanted with BCR-ABL infected control Smo+/+ or Smo−/− cells. Cells were analyzed by flow cytometry for lin-GFP+ cells and subsequently for c-Kit+Sca+ cells that define the CML stem cell population. GFP reflects BCR-ABL expression. FIG. 2, panel (c) is a graph of the average percentage of CML stem cells in mice receiving BCR-ABL infected control Smo+/+ or Smo−/− KLS cells (n=8 for Smo+/+ and n=9 for Smo−/−, p=0.006).

### Example 3

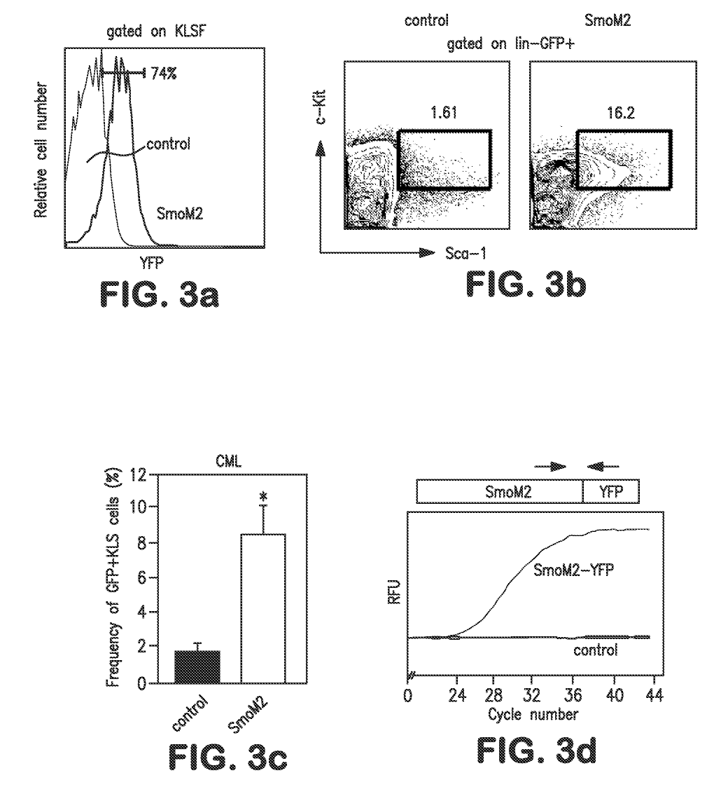

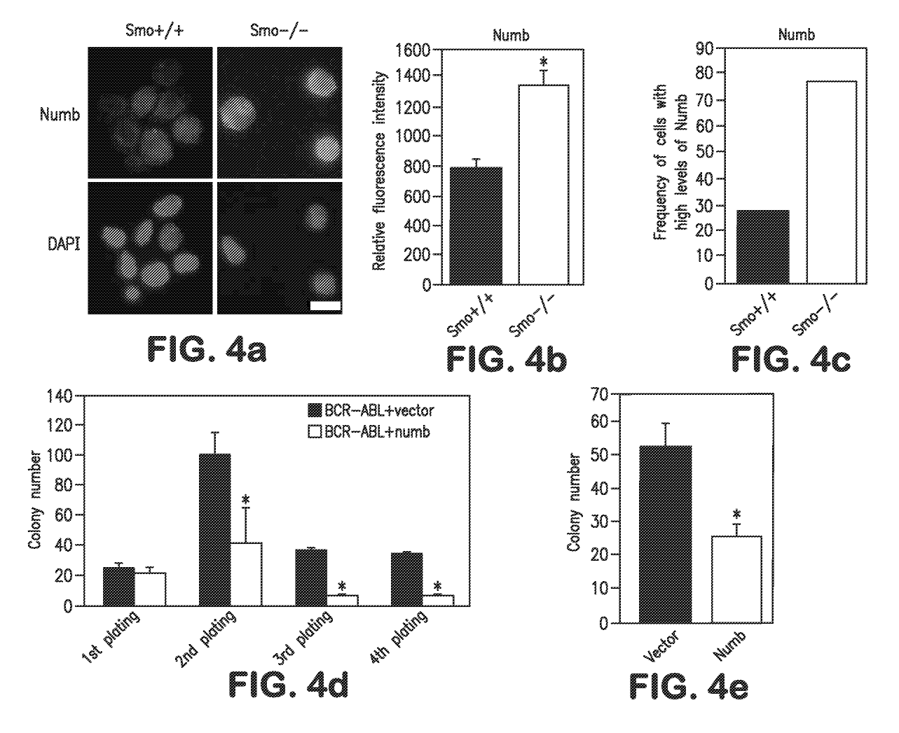

To investigate whether activation of the Hh pathway might lead to an increased frequency of CML stem cells, we examined a transgenic mouse expressing an activated, mutant form of Smo in the hematopoietic system. This mutant form of the Smo protein is fused to yellow fluorescent protein (YFP) and has an oncogenic missense alteration (W539L) which renders this mutant Smo protein insensitive to inhibition by Ptch. Expression of the Smo-EYFP fusion gene is blocked by a loxP-flanked STOP fragment placed between the ROSA promoter and the Smo/EYFP sequence. The transgenic mouse (SmoM2) was crossed with the Vav-Cre transgenic which allowed deletion of the STOP sequence and thus expression of the SmoM2-EYFP fusion protein in the hematopoietic system. Following Cre mediated deletion SmoM2 expression is reflected by the expression of YFP. We isolated KLSF cells from these mice and found that YFP was detectable in over 70% percent of the cells, thus confirming expression of SmoM2 (FIG. 3, panel (a)).

To determine whether constitutive activation of Smo enhances CML stem cell growth, we isolated control and SmoM2 KLSF cells, infected with BCR-ABL1, and transplanted these cells into irradiated mice. While both cell types supported development of CML, the frequency of CML stem cells in animals receiving SmoM2 cells was four fold greater as compared to control (FIG. 3, panel (b); and FIG. 3, panel (c)). As expected, SmoM2 was expressed in the transgenic CML stem cells, but not control CML stem cells (FIG. 3, panel (d)). These data cumulatively show that Hh pathway activity controls the frequency and maintenance of CML stem cells.

Additional details concerning the experiment results shown in FIG. 3 are as described here. FIG. 3 shows the presence of constitutively active Smoothened increases the frequency of CML stem cells. FIG. 3, panel (a) shows the KLSF population in bone marrow cells from control and SmoM2 mice as analyzed by FACS for YFP fluorescence. YFP fluorescence reflects SmoM2 expression levels. The histogram is a representative of four experiments. FIG. 3, panel (b) shows CML cell numbers from mice transplanted with BCR-ABL1 infected control (left) and constitutively activated Smoothened (SmoM2, right). Bone marrow KLSF cells were analyzed for the presence of CML stem cells (KLS cells) by staining for lin-GFP+ cells, and subsequently for c-Kit+Sca+ cells. FIG. 3, panel (c) is a graph of the average percentage of CML stem cells (GFP+KLS cells) in mice receiving BCR-ABL transduced control and SmoM2 KLSF cells (n=4 for control and n=12 for SmoM2, p=0.048). FIG. 3, panel (d) shows a real-time PCR analysis of SmoM2 expression in CML stem cells (GFP+KLS).

### Example 4

Maintenance of undifferentiated hematopoietic stem cells depends on low levels of the cell fate determinant, Numb, as overexpression of Numb can impose a differentiated state and deplete the immature stem cell population.

We tested whether Numb has a similar function in maintenance of CML stem cells, and whether the effects of loss of Smo may be due to altered levels of Numb. We found that a greater frequency of Smo−/−CML KLS cells had high levels of Numb as compared to control CML KLS cells (FIG. 4, panel (a); FIG. 4, panel (b); FIG. 4, panel (c)).

To determine whether high levels of Numb have the ability to inhibit CML growth, we carried out a serial replating assay of BCR-ABL1 infected KLS cells in the presence or absence of Numb. The presence of BCR-ABL1 led to the growth of colonies which was sustained through four replatings (FIG. 4, panel (d)). However, the additional presence of Numb caused a significant reduction starting at the second plating and continuing through the fourth plating (FIG. 4, panel (d)). Finally, isolated cancer stem cells from fully formed CML infected with either vector control or Numb revealed that Numb could suppress the propagation of CML stem cells (FIG. 4, panel (e)). These data show that the increased expression of Numb contributes functionally to the loss of CML stem cells in the absence of Smo.

Additional details concerning the experiment results shown in FIG. 4 are as described here. FIG. 4 shows that loss of Smoothened increases the frequency of cells with high levels of Numb and contributes to decreased CML growth. FIG. 4, panel (a) shows increased levels of Numb expression in Smoothened null cells. CML stem cells from control (Smo+/+), and Smoothened null (Smo−/−) leukemia cells were sorted, cytospun, and stained with Numb (red, upper panel) and DAPI (blue, lower panel). In FIG. 4, panel (b) and FIG. 4, panel (c) the mean fluorescence intensity of Numb expression was quantified using METAMORPH® software. Average intensity per pixel was determined by dividing the overall fluorescence intensity by the area of the cell. In FIG. 4, panel (c) the frequency of cells with high expression of Numb was calculated by designating cells above the mean fluorescence intensity value of 1,000 as high expressors. Data shown is representative of three independent experiments using either CML KLS or CML c-Kit+ cells when cancer stem cell numbers were limiting in the Smo−/−. FIG. 4, panel (d) shows that ectopic expression of Numb inhibits long term serial replating of BCR-ABL1 transformed cells. Bone marrow KLSF cells were infected with either BCR-ABL-IRES-GFP and vector-IRES-YFP or BCR-ABL-IRES-GFP and Numb-IRES-YFP and 1,000 GFP/YFP double positive cells were plated into methylcellulose media (M3434) to assess primary colony formation. Colony numbers were counted on days 8-10. Cells were then harvested, counted and 10,000 cells were replated for a second, third and fourth time and colonies counted on day 8-10 after each replating. Error bars show s.e.m. Note that from second replating, Numb infected cells showed significantly reduced colony forming ability (2^{nd} plating P=0.0067, 3^{rd} plating P=0.006 and 4^{th} plating P=0.002). FIG. 4, panel (e) shows a graph of colony numbers when CML KLS cells were infected with viruses expressing control vector or Numb IRES-YFP, double positive cells sorted and plated in methylcellulose media. Colonies were counted after 7-10 days. The graph shows representative results from two independent experiments. P=0.03.

### Example 5

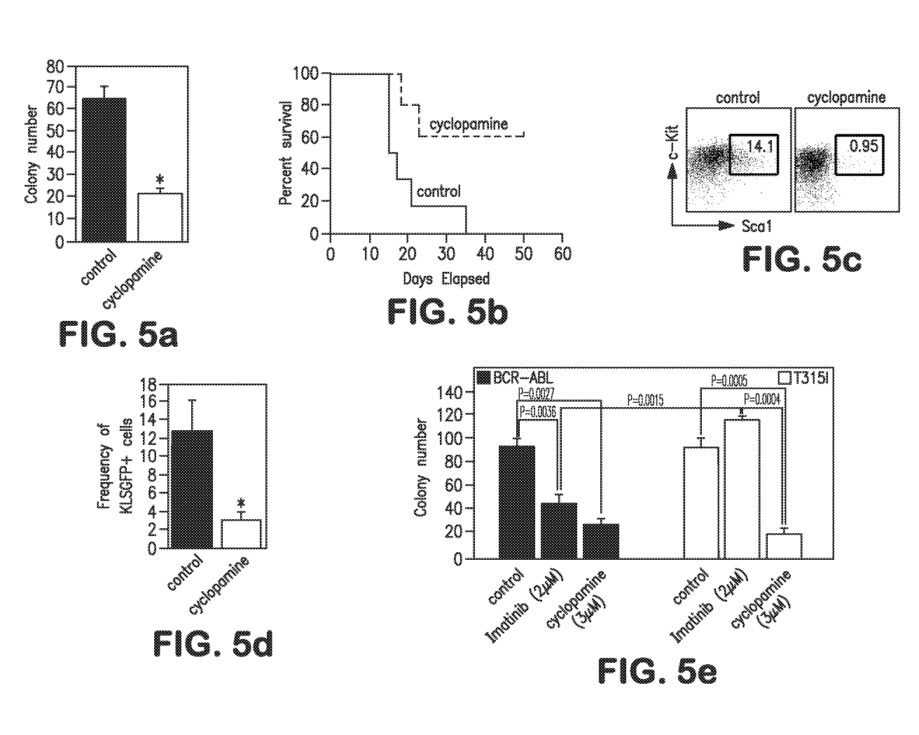

Our demonstration that CML stem cells are dependent on Hh pathway activity raised the possibility that these cells might be targeted by pharmacological blockade of this pathway. We therefore tested the effect of cyclopamine, a plant-derived alkaloid that inhibits Hh signaling by binding and stabilizing Smoothened in its inactive form. We first tested whether cyclopamine could inhibit the growth of CML cells in vitro. Fractionated cells from fully developed leukemia cells were plated in methylcellulose in the presence or absence of cyclopamine. While fractionated CML stem cells grew robustly in vitro under control conditions, exposure to cyclopamine led to a two-fold inhibition of colony growth (FIG. 5, panel (a)) demonstrating that cyclopamine could inhibit BCR-ABL1-driven growth in vitro.

To test if cyclopamine could also inhibit growth of BCR-ABL1 driven leukemia in vivo, we infected KLSF cells from wild-type mice with BCR-ABL and transplanted them into lethally irradiated recipients. Following transplantation, cyclopamine was delivered via oral gavage, and mouse survival was analyzed. Over a period of four weeks, 100% of control animals succumbed to CML, whereas 40% of mice treated with cyclopamine were still alive after seven weeks (FIG. 5, panel (b)). Analysis of the leukemias in mice treated with cyclopamine revealed up to a fourteen-fold reduction in the CML stem cell population (FIG. 5, panel (c); and FIG. 5, panel (d)). These data show that cyclopamine can target the CML stem cell population critical for the propagation of CML.

One therapy for CML involves use of the tyrosine kinase inhibitor imatinib mesylate. However, the long term effectiveness of this inhibitor is limited because cells eventually arise carrying mutations in BCR-ABL1 which render these cells resistant to imatinib and leads to the development of imatinib-resistant CML. Targeting pathways involved in CML, such as the Hh signal transduction pathway, may complement use of imatinib and prevent or overcome imatinib resistance.

We thus tested whether cyclopamine could impair the growth of imatinib resistant CML. To this end we infected cells with virus encoding either wild type BCR-ABL1, or an imatinib resistant T315I mutant, and tracked the growth of these cells upon treatment with imatinib or cyclopamine. The growth of cells with control BCR-ABL1 was inhibited in response to imatinib treatment, whereas cells carrying the mutant BCR-ABL T3151 protein remained unresponsive to imatinib treatment (FIG. 5, panel (e).) Remarkably, however, growth driven by either control BCR-ABL1 or mutant BCR-ABL T315I was reduced 6-fold in the presence of cyclopamine. These data show that Hh pathway activity is required for the maintenance of CML leukemia stem cells. These data also show small molecule inhibitors of the Hh pathway can be used to control and treat both normal and imatinib-resistant CML.

Additional details concerning the experiment results shown in FIG. 5 are as described here. FIG. 5 shows that inhibition of Smoothened by cyclopamine delivery impairs CML development and progression. In FIG. 5, panel (a) 1,000 KLS GFP+ cells were sorted from CML and plated in methylcellulose media (M3434) in the presence of 3 μM tomatidine (control) or cyclopamine. Colonies were counted after 7-10 days. Data shown is representative of two independent experiments (P=0.005). FIG. 5, panel (b) shows a survival curve for mice receiving a transplant of 20,000 BCR-ABL1 infected KLS cells in the presence of vehicle or 25 mg/kg of cyclopamine. Following transplantation, cyclopamine was delivered via gavage as described in methods, and mice monitored for a total of 50 days. Results are representative of two experiments with KLS and one experiment with 5-fluorouracil (5FU) treated whole bone marrow using a total of 32 mice over three experiments. P=0.02. FIG. 5, panel (c) shows a representative example of the frequency of CML stem cells in CML treated with cyclopamine or vehicle control. Cells were analyzed by flow cytometry for lin-GFP+ cells, and subsequently for c-Kit⁺Sca-1⁺ cells that define the CML stem cell population (left, control, right, cyclopamine treated). FIG. 5, panel (d) shows the average frequency of CML stem cells in CML treated with cyclopamine or vehicle control. (n=4, P=0.03). In FIG. 5, panel (e) bone marrow KLSF cells were infected with wild type BCR-ABL or imatinib resistant mutant of BCR-ABL (BCR-ABL T315I) and 600 GFP positive cells were sorted into wells containing methylcellulose media to assess colony forming ability in the presence or absence of imatinib (2 μM) and/or cyclopamine (3 μM). Colony numbers were counted on days 8-10. The graph shown is representative of two independent experiments. Error bars show s.e.m. Note that when treated with imatinib, the colony number from wild type BCR-ABL infected cells was significantly reduced compared to T315I mutant infected cells. P=0.0015. In addition, the colony number from cyclopamine treated T315I mutant infected cells was significantly lower than the colony number from imatinib treated T315I mutant infected cells.

The present disclosure now being fully described, it will be apparent to one of ordinary skill in the art that many changes and modifications can be made thereto without departing from the spirit or scope of the appended claims.

What is claimed is:

1. A method of treating a hematopoietic cell malignancy in a subject comprising administering a therapeutically effective amount of an Hh antagonist to a subject with a hematopoietic cell malignancy; whereby the hematopoietic cell malignancy is treated.

2. The method of claim 1 wherein the Hh antagonist is selected from the group consisting of cyclopamine, cyclopamine salts and arsenic trioxide.

3. A method of treating chronic myelogenous leukemia in a subject comprising administering a therapeutically effective amount of an Hh antagonist to a subject with chronic myelogenous leukemia; whereby the chronic myelogenous leukemia is treated.

4. The method of claim 3 wherein the Hh antagonist is selected from the group consisting of cyclopamine, cyclopamine salts and arsenic trioxide.

5. A method of treating chronic myelogenous leukemia in a subject comprising administering a therapeutically effective amount of a Smo antagonist to a subject with chronic myelogenous leukemia; whereby the chronic myelogenous leukemia is treated.

6. The method of claim 5 wherein the Smo antagonist is selected from the group consisting of cyclopamine and cyclopamine salts.

7. The method of claim 5 wherein the Smo antagonist is cyclopamine.

8. A method of treating a hematopoietic cell malignancy in a subject comprising administering a therapeutically effective amount of an Hh antagonist and a therapeutically effective amount of a BCR-ABL antagonist to a subject with a hematopoietic cell malignancy; whereby the hematopoietic cell malignancy is treated.

9. The method of claim 8 wherein the Hh antagonist is selected from the group consisting of cyclopamine, cyclopamine salts and arsenic trioxide.

10. The method of claim 8 wherein the BCR-ABL antagonist is selected from the group consisting of imantinib, imatinib mesilate, dasatinib and nilotinib.

11. A method of treating chronic myelogenous leukemia in a subject comprising administering a therapeutically effective amount of an Hh antagonist and a therapeutically effective amount of a BCR-ABL antagonist to a subject with chronic myelogenous leukemia; whereby the chronic myelogenous leukemia is treated.

12. The method of claim 11 wherein the Hh antagonist is selected from the group consisting of cyclopamine, cyclopamine salts and arsenic trioxide; and the BCR-ABL antagonist is selected from the group consisting of imantinib, imatinib mesilate, dasatinib and nilotinib.

13. A method of treating chronic myelogenous leukemia in a subject comprising administering a therapeutically effective amount of a Smo antagonist and a therapeutically effective amount of a BCR-ABL antagonist to a subject with chronic myelogenous leukemia; whereby the chronic myelogenous leukemia is treated.

14. The method of claim 13 wherein the Smo antagonist is selected from the group consisting of cyclopamine and cyclopamine salts; and the BCR-ABL antagonist is selected from the group consisting of imantinib, imatinib mesilate, dasatinib and nilotinib.

15. The method of claim 13 wherein the Smo antagonist is cyclopamine and the BCR-ABL antagonist is imantinib.

16. A method of decreasing the number of hematopoietic malignancy stem cells in a tissue of a subject comprising administering a therapeutically effective amount of an Hh antagonist to a subject having a number of hematopoietic malignancy stem cells in a tissue; whereby the number of hematopoietic malignancy stem cells in the tissue of the subject is decreased.

17. The method of claim 16 wherein the Hh antagonist is selected from the group consisting of cyclopamine, cyclopamine salts and arsenic trioxide.

18. A method of decreasing the number of chronic myelogenous leukemia stem cells in a tissue of a subject comprising administering a therapeutically effective amount of an Hh antagonist to a subject having a number of chronic myelogenous leukemia stem cells in a tissue; whereby the number of chronic myelogenous leukemia stem cells in the tissue of the subject is decreased.

19. The method of claim 18 wherein the Hh antagonist is selected from the group consisting of cyclopamine, cyclopamine salts and arsenic trioxide.

20. A method of decreasing the number of chronic myelogenous leukemia stem cells in a tissue of a subject comprising administering a therapeutically effective amount of a Smo antagonist to a subject having a number of chronic myelogenous leukemia stem cells in a tissue; whereby the number of chronic myelogenous leukemia stem cells in the tissue of the subject is decreased.

21. The method of claim 20 wherein the Smo antagonist is selected from the group consisting of cyclopamine and cyclopamine salts.

22. The method of claim 20 wherein the Smo antagonist is cyclopamine.

23. A method of decreasing the number hematopoietic malignancy stem cells in a tissue of a subject comprising administering a therapeutically effective amount of an Hh antagonist and a therapeutically effective amount of a BCR-ABL antagonist to a subject having a number of hematopoietic malignancy stem cells in a tissue; whereby the number of hematopoietic malignancy stem cells in the tissue of the subject is decreased.

24. The method of claim 23 wherein the Hh antagonist is selected from the group consisting of cyclopamine, cyclopamine salts and arsenic trioxide.

25. The method of claim 23 wherein the BCR-ABL antagonist is selected from the group consisting of imantinib, imatinib mesilate, dasatinib and nilotinib.

26. A method of decreasing the number of chronic myelogenous leukemia stem cells in a tissue of a subject comprising administering a therapeutically effective amount of an Hh antagonist and a therapeutically effective amount of a BCR-ABL antagonist to a subject having a number of chronic myelogenous leukemia stem cells in a tissue; whereby the number of chronic myelogenous leukemia stem cells in the tissue of the subject is decreased.

27. The method of claim 26 wherein the Hh antagonist is selected from the group consisting of cyclopamine, cyclopamine salts and arsenic trioxide; and the BCR-ABL antagonist is selected from the group consisting of imantinib, imatinib mesilate, dasatinib and nilotinib.

28. A method of decreasing the number of chronic myelogenous leukemia stem cells in a tissue of a subject comprising administering a therapeutically effective amount of a Smo antagonist and a therapeutically effective amount of a BCR-ABL antagonist to a subject having a number of chronic myelogenous leukemia stem cells in a tissue; whereby the number of chronic myelogenous leukemia stem cells in the tissue of the subject is decreased.

29. The method of claim 28 wherein the Smo antagonist is selected from the group consisting of cyclopamine and cyclopamine salts; and the BCR-ABL antagonist is selected from the group consisting of imantinib, imatinib mesilate, dasatinib and nilotinib.

30. The method of claim 28 wherein the Smo antagonist is cyclopamine and the BCR-ABL antagonist is imantinib.


SEQUENCE LISTING (16 sequences)

SEQ ID NO: 1 - protein, 436 aa, Mus musculus
LLLLARCFLVILASSLLVCPGLACGPGRGFGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLRPGDRVLAADDQGRLLYSDFLTFLDRDEGAKKVFYVIETLEPRERLLLTAAHLLFVAPHNDSGPTPGPSALFASRVRPGQRVYVVAERGGDRRLLPAAVHSVTLREEEAGAYAPLTAHGTILINRVLASCYAVIEEHSWAHRAFAPFRLAHALLAALAPARTDGGGGGSIPAAQSATEARGAEPTAGIHWYSQLLYHIGTWLLDSETMHPLGMAVKSS

SEQ ID NO: 2 - protein, 461 aa, Homo sapiens
LLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS

SEQ ID NO: 3 - protein, 448 aa, Mus musculus
ESPRATQTPESPKLSQPRAHLSAHQAPSPAALPGYPAMSPAWLRPRLRFCLFLLLLLLVPAARGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLENGERVALSAVKPGDRVLAMGEDGTPTFSDVLIFLDREPNRLRAFQVIETQDPPRRLALTPAHLLFIADNHTEPAAHFRATFASHVQPGQYVLVSGVPGLQPARVAAVSTHVALGSYAPLTRHGTLVVEDVVASCFAAVADHHLAQLAFWPLRLFPSLAWGSWTPSEGVHWYPQMLYRLGRLLLEESTFHPLGMSGAGS

SEQ ID NO: 4 - protein, 410 aa, Homo sapiens
SPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 5 - protein, 395 aa, Mus musculus
ALPASLLPLCCLALLALSAQSCGPGRGPVGRRRYVRKQLVPLLYKQFVPSMPERTLGASGPAEGRVTRGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHIHVSVKADNSLAVRAGGCFPGNATVRLRSGERKGLRELHRGDWVLAADAAGRVVPTPVLLFLDRDLQRRASFVAVETERPPRKLLLTPWHLVFAARGPAPAPGDFAPVFARRLRAGDSVLAPGGDALQPARVARVAREEAVGVFAPLTAHGTLLVNDVLASCYAVLESHQWAHRAFAPLRLLHALGALLPGGAVQPTGMHWYSRLLYRLAEELMG

SEQ ID NO: 6 - protein, 395 aa, Homo sapiens
ALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 7 - protein, 1433 aa, Mus musculus
ASAGNAAGALGRQAGGGRRRRTGGPHRAAPDRDYLHRPSYCDAAFALEQISKGKATGRKAPLWLRAKFQRLLFKLGCYIQKNCGKFLVVGLLIFGAFAVGLKAANLETNVEELWVEVGGRVSRELNYTRQKIGEEAMFNPQLMIQTPKEEGANVLTTEALLQHLDSALQASRVHVYMYNRQWKLEHLCYKSGELITETGYMDQIIEYLYPCLIITPLDCFWEGAKLQSGTAYLLGKPPLRWTNFDPLEFLEELKKINYQVDSWEEMLNKAEVGHGYMDRPCLNPADPDCPATAPNKNSTKPLDVALVLNGGCQGLSRKYMHWQEELIVGGTVKNATGKLVSAHALQTMFQLMTPKQMYEHFRGYDYVSHINWNEDRAAAILEAWQRTYVEVVHQSVAPNSTQKVLPFTTTTLDDILKSFSDVSVIRVASGYLLMLAYACLTMLRWDCSKSQGAVGLAGVLLVALSVAAGLGLCSLIGISFNAATTQVLPFLALGVGVDDVFLLAHAFSETGQNKRIPFEDRTGECLKRTGASVALTSISNVTAFFMAALIPIPALRAFSLQAAVVVVFNFAMVLLIFPAILSMDLYRREDRRLDIFCCFTSPCVSRVIQVEPQAYTEPHSNTRYSPPPPYTSHSFAHETHITMQSTVQLRTEYDPHTHVYYTTAEPRSEISVQPVTVTQDNLSCQSPESTSSTRDLLSQFSDSSLHCLEPPCTKWTLSSFAEKHYAPFLLKPKAKVVVILLFLGLLGVSLYGTTRVRDGLDLTDIVPRETREYDFIAAQFKYFSFYNMYIVTQKADYPNIQHLLYDLHKSFSNVKYVMLEENKQLPQMWLHYFRDWLQGLQDAFDSDWETGRIMPNNYKNGSDDGVLAYKLLVQTGSRDKPIDISQLTKQRLVDADGIINPSAFYIYLTAWVSNDPVAYAASQANIRPHRPEWVHDKADYMPETRLRIPAAEPIEYAQFPFYLNGLRDTSDFVEAIEKVRVICNNYTSLGLSSYPNGYPFLFWEQYISLRHWLLLSISVVLACTFLVCAVFLLNPWTAGIIVMVLALMTVELFGMMGLIGIKLSAVPVVILIASVGIGVEFTVHVALAFLTAIGDKNHRAMLALEHMFAPVLDGAVSTLLGVLMLAGSEFDFIVRYFFAVLAILTVLGVLNGLVLLPVLLSFFGPCPEVSPANGLNRLPTPSPEPPPSVVRFAVPPGHTNNGSDSSDSEYSSQTTVSGISEELRQYEAQQGAGGPAHQVIVEATENPVFARSTVVHPDSRHQPPLTPRQQPHLDSGSLSPGRQGQQPRRDPPREGLRPPPYRPRRDAFEISTEGHSGPSNRDRSGPRGARSHNPRNPTSTAMGSSVPSYCQPITTVTASASVTVAVHPPPGPGRNPRGGPCPGYESYPETDHGVFEDPHVPFHVRCERRDSKVEVIELQDVECEERPWGSSSN

SEQ ID NO: 8 - protein, 1446 aa, Homo sapiens
ASAGNAAEPQDRGGGGSGCIGAPGRPAGGGRRRRTGGLRRAAAPDRDYLHRPSYCDAAFALEQISKGKATGRKAPLWLRAKFQRLLFKLGCYIQKNCGKFLVVGLLIFGAFAVGLKAANLETNVEELWVEVGGRVSRELNYTRQKIGEEAMFNPQLMIQTPKEEGANVLTTEALLQHLDSALQASRVHVYMYNRQWKLEHLCYKSGELITETGYMDQIIEYLYPCLIITPLDCFWEGAKLQSGTAYLLGKPPLRWTNFDPLEFLEELKKINYQVDSWEEMLNKAEVGHGYMDRPCLNPADPDCPATAPNKNSTKPLDMALVLNGGCHGLSRKYMHWQEELIVGGTVKNSTGKLVSAHALQTMFQLMTPKQMYEHFKGYEYVSHINWNEDKAAAILEAWQRTYVEVVHQSVAQNSTQKVLSFTTTTLDDILKSFSDVSVIRVASGYLLMLAYACLTMLRWDCSKSQGAVGLAGVLLVALSVAAGLGLCSLIGISFNAATTQVLPFLALGVGVDDVFLLAHAFSETGQNKRIPFEDRTGECLKRTGASVALTSISNVTAFFMAALIPIPALRAFSLQAAVVVVFNFAMVLLIFPAILSMDLYRREDRRLDIFCCFTSPCVSRVIQVEPQAYTDTHDNTRYSPPPPYSSHSFAHETQITMQSTVQLRTEYDPHTHVYYTTAEPRSEISVQPVTVTQDTLSCQSPESTSSTRDLLSQFSDSSLHCLEPPCTKWTLSSFAEKHYAPFLLKPKAKVVVIFLFLGLLGVSLYGTTRVRDGLDLTDIVPRETREYDFIAAQFKYFSFYNMYIVTQKADYPNIQHLLYDLHRSFSNVKYVMLEENKQLPKMWLHYFRDWLQGLQDAFDSDWETGKIMPNNYKNGSDDGVLAYKLLVQTGSRDKPIDISQLTKQRLVDADGIINPSAFYIYLTAWVSNDPVAYAASQANIRPHRPEWVHDKADYMPETRLRIPAAEPIEYAQFPFYLNGLRDTSDFVEAIEKVRTICSNYTSLGLSSYPNGYPFLFWEQYIGLRHWLLLFISVVLACTFLVCAVFLLNPWTAGIIVMVLALMTVELFGMMGLIGIKLSAVPVVILIASVGIGVEFTVHVALAFLTAIGDKNRRAVLALEHMFAPVLDGAVSTLLGVLMLAGSEFDFIVRYFFAVLAILTILGVLNGLVLLPVLLSFFGPYPEVSPANGLNRLPTPSPEPPPSVVRFAMPPGHTHSGSDSSDSEYSSQTTVSGLSEELRHYEAQQGAGGPAHQVIVEATENPVFAHSTVVHPESRHHPPSNPRQQPHLDSGSLPPGRQGQQPRRDPPREGLWPPPYRPRRDAFEISTEGHSGPSNRARWGPRGARSHNPRNPASTAMGSSVPGYCQPITTVTASASVTVAVHPPPVPGPGRNPRGGLCPGYPETDHGLFEDPHVPFHVRCERRDSKVEVIELQDVECEERPRGSSSN

SEQ ID NO: 9 - protein, 1445 aa, Homo sapiens
ELLNRNRLVIVSPRCTPPKASGGPARRGFYTFRSFCKDGGGGEEEEENGGEEKDDRGDKETRSDKGKATGRKAPLWLRAKFQRLLFKLGCYIQKNCGKFLVVGLLIFGAFAVGLKAANLETNVEELWVEVGGRVSRELNYTRQKIGEEAMFNPQLMIQTPKEEGANVLTTEALLQHLDSALQASRVHVYMYNRQWKLEHLCYKSGELITETGYMDQIIEYLYPCLIITPLDCFWEGAKLQSGTAYLLGKPPLRWTNFDPLEFLEELKKINYQVDSWEEMLNKAEVGHGYMDRPCLNPADPDCPATAPNKNSTKPLDMALVLNGGCHGLSRKYMHWQEELIVGGTVKNSTGKLVSAHALQTMFQLMTPKQMYEHFKGYEYVSHINWNEDKAAAILEAWQRTYVEVVHQSVAQNSTQKVLSFTTTTLDDILKSFSDVSVIRVASGYLLMLAYACLTMLRWDCSKSQGAVGLAGVLLVALSVAAGLGLCSLIGISFNAATTQVLPFLALGVGVDDVFLLAHAFSETGQNKRIPFEDRTGECLKRTGASVALTSISNVTAFFMAALIPIPALRAFSLQAAVVVVFNFAMVLLIFPAILSMDLYRREDRRLDIFCCFTSPCVSRVIQVEPQAYTDTHDNTRYSPPPPYSSHSFAHETQITMQSTVQLRTEYDPHTHVYYTTAEPRSEISVQPVTVTQDTLSCQSPESTSSTRDLLSQFSDSSLHCLEPPCTKWTLSSFAEKHYAPFLLKPKAKVVVIFLFLGLLGVSLYGTTRVRDGLDLTDIVPRETREYDFIAAQFKYFSFYNMYIVTQKADYPNIQHLLYDLHRSFSNVKYVMLEENKQLPKMWLHYFRDWLQGLQDAFDSDWETGKIMPNNYKNGSDDGVLAYKLLVQTGSRDKPIDISQLTKQRLVDADGIINPSAFYIYLTAWVSNDPVAYAASQANIRPHRPEWVHDKADYMPETRLRIPAAEPIEYAQFPFYLNGLRDTSDFVEAIEKVRTICSNYTSLGLSSYPNGYPFLFWEQYIGLRHWLLLFISVVLACTFLVCAVFLLNPWTAGIIVMVLALMTVELFGMMGLIGIKLSAVPVVILIASVGIGVEFTVHVALAFLTAIGDKNRRAVLALEHMFAPVLDGAVSTLLGVLMLAGSEFDFIVRYFFAVLAILTILGVLNGLVLLPVLLSFFGPYPEVSPANGLNRLPTPSPEPPPSVVRFAMPPGHTHSGSDSSDSEYSSQTTVSGLSEELRHYEAQQGAGGPAHQVIVEATENPVFAHSTVVHPESRHHPPSNPRQQPHLDSGSLPPGRQGQQPRRDPPREGLWPPPYRPRRDAFEISTEGHSGPSNRARWGPRGARSHNPRNPASTAMGSSVPGYCQPITTVTASASVTVAVHPPPVPGPGRNPRGGLCPGYPETDHGLFEDPHVPFHVRCERRDSKVEVIELQDVECEERPRGSSSN

SEQ ID NO: 10 - protein, 1380 aa, Homo sapiens
GKATGRKAPLWLRAKFQRLLFKLGCYIQKNCGKFLVVGLLIFGAFAVGLKAANLETNVEELWVEVGGRVSRELNYTRQKIGEEAMFNPQLMIQTPKEEGANVLTTEALLQHLDSALQASRVHVYMYNRQWKLEHLCYKSGELITETGYMDQIIEYLYPCLIITPLDCFWEGAKLQSGTAYLLGKPPLRWTNFDPLEFLEELKKINYQVDSWEEMLNKAEVGHGYMDRPCLNPADPDCPATAPNKNSTKPLDMALVLNGGCHGLSRKYMHWQEELIVGGTVKNSTGKLVSAHALQTMFQLMTPKQMYEHFKGYEYVSHINWNEDKAAAILEAWQRTYVEVVHQSVAQNSTQKVLSFTTTTLDDILKSFSDVSVIRVASGYLLMLAYACLTMLRWDCSKSQGAVGLAGVLLVALSVAAGLGLCSLIGISFNAATTQVLPFLALGVGVDDVFLLAHAFSETGQNKRIPFEDRTGECLKRTGASVALTSISNVTAFFMAALIPIPALRAFSLQAAVVVVFNFAMVLLIFPAILSMDLYRREDRRLDIFCCFTSPCVSRVIQVEPQAYTDTHDNTRYSPPPPYSSHSFAHETQITMQSTVQLRTEYDPHTHVYYTTAEPRSEISVQPVTVTQDTLSCQSPESTSSTRDLLSQFSDSSLHCLEPPCTKWTLSSFAEKHYAPFLLKPKAKVVVIFLFLGLLGVSLYGTTRVRDGLDLTDIVPRETREYDFIAAQFKYFSFYNMYIVTQKADYPNIQHLLYDLHRSFSNVKYVMLEENKQLPKMWLHYFRDWLQGLQDAFDSDWETGKIMPNNYKNGSDDGVLAYKLLVQTGSRDKPIDISQLTKQRLVDADGIINPSAFYIYLTAWVSNDPVAYAASQANIRPHRPEWVHDKADYMPETRLRIPAAEPIEYAQFPFYLNGLRDTSDFVEAIEKVRTICSNYTSLGLSSYPNGYPFLFWEQYIGLRHWLLLFISVVLACTFLVCAVFLLNPWTAGIIVMVLALMTVELFGMMGLIGIKLSAVPVVILIASVGIGVEFTVHVALAFLTAIGDKNRRAVLALEHMFAPVLDGAVSTLLGVLMLAGSEFDFIVRYFFAVLAILTILGVLNGLVLLPVLLSFFGPYPEVSPANGLNRLPTPSPEPPPSVVRFAMPPGHTHSGSDSSDSEYSSQTTVSGLSEELRHYEAQQGAGGPAHQVIVEATENPVFAHSTVVHPESRHHPPSNPRQQPHLDSGSLPPGRQGQQPRRDPPREGLWPPPYRPRRDAFEISTEGHSGPSNRARWGPRGARSHNPRNPASTAMGSSVPGYCQPITTVTASASVTVAVHPPPVPGPGRNPRGGLCPGYPETDHGLFEDPHVPFHVRCERRDSKVEVIELQDVECEERPRGSSSN

SEQ ID NO: 11 - protein, 1295 aa, Homo sapiens
FNPQLMIQTPKEEGANVLTTEALLQHLDSALQASRVHVYMYNRQWKLEHLCYKSGELITETGYMDQIIEYLYPCLIITPLDCFWEGAKLQSGTAYLLGKPPLRWTNFDPLEFLEELKKINYQVDSWEEMLNKAEVGHGYMDRPCLNPADPDCPATAPNKNSTKPLDMALVLNGGCHGLSRKYMHWQEELIVGGTVKNSTGKLVSAHALQTMFQLMTPKQMYEHFKGYEYVSHINWNEDKAAAILEAWQRTYVEVVHQSVAQNSTQKVLSFTTTTLDDILKSFSDVSVIRVASGYLLMLAYACLTMLRWDCSKSQGAVGLAGVLLVALSVAAGLGLCSLIGISFNAATTQVLPFLALGVGVDDVFLLAHAFSETGQNKRIPFEDRTGECLKRTGASVALTSISNVTAFFMAALIPIPALRAFSLQAAVVVVFNFAMVLLIFPAILSMDLYRREDRRLDIFCCFTSPCVSRVIQVEPQAYTDTHDNTRYSPPPPYSSHSFAHETQITMQSTVQLRTEYDPHTHVYYTTAEPRSEISVQPVTVTQDTLSCQSPESTSSTRDLLSQFSDSSLHCLEPPCTKWTLSSFAEKHYAPFLLKPKAKVVVIFLFLGLLGVSLYGTTRVRDGLDLTDIVPRETREYDFIAAQFKYFSFYNMYIVTQKADYPNIQHLLYDLHRSFSNVKYVMLEENKQLPKMWLHYFRDWLQGLQDAFDSDWETGKIMPNNYKNGSDDGVLAYKLLVQTGSRDKPIDISQLTKQRLVDADGIINPSAFYIYLTAWVSNDPVAYAASQANIRPHRPEWVHDKADYMPETRLRIPAAEPIEYAQFPFYLNGLRDTSDFVEAIEKVRTICSNYTSLGLSSYPNGYPFLFWEQYIGLRHWLLLFISVVLACTFLVCAVFLLNPWTAGIIVMVLALMTVELFGMMGLIGIKLSAVPVVILIASVGIGVEFTVHVALAFLTAIGDKNRRAVLALEHMFAPVLDGAVSTLLGVLMLAGSEFDFIVRYFFAVLAILTILGVLNGLVLLPVLLSFFGPYPEVSPANGLNRLPTPSPEPPPSVVRFAMPPGHTHSGSDSSDSEYSSQTTVSGLSEELRHYEAQQGAGGPAHQVIVEATENPVFAHSTVVHPESRHHPPSNPRQQPHLDSGSLPPGRQGQQPRRDPPREGLWPPPYRPRRDAFEISTEGHSGPSNRARWGPRGARSHNPRNPASTAMGSSVPGYCQPITTVTASASVTVAVHPPPVPGPGRNPRGGLCPGYPETDHGLFEDPHVPFHVRCERRDSKVEVIELQDVECEERPRGSSSN

SEQ ID NO: 12 - protein, 792 aa, Mus musculus
AAGRPVRGPELAPRRLLQLLLLVLLGGPGRGAALSGNVTGPGPHSASGSSRRDVPVTSPPPPLLSHCGRAAHCEPLRYNVCLGSALPYGATTTLLAGDSDSQEEAHGKLVLWSGLRNAPRCWAVIQPLLCAVYMPKCENDRVELPSRTLCQATRGPCAIVERERGWPDFLRCTPDHFPEGCPNEVQNIKFNSSGQCEAPLVRTDNPKSWYEDVEGCGIQCQNPLFTEAEHQDMHSYIAAFGAVTGLCTLFTLATFVADWRNSNRYPAVILFYVNACFFVGSIGWLAQFMDGARREIVCRADGTMRFGEPTSSETLSCVIIFVIVYYALMAGVVWFVVLTYAWHTSFKALGTTYQPLSGKTSYFHLLTWSLPFVLTVAILAVAQVDGDSVSGICFVGYKNYRYRAGFVLAPIGLVLIVGGYFLIRGVMTLFSIKSNHPGLLSEKAASKINETMLRLGIFGFLAFGFVLITFSCHFYDFFNQAEWERSFRDYVLCQANVTIGLPTKKPIPDCEIKNRPSLLVEKINLFAMFGTGIAMSTWVWTKATLLIWRRTWCRLTGHSDDEPKRIKKSKMIAKAFSKRRELLQNPGQELSFSMHTVSHDGPVAGLAFDLNEPSADVSSAWAQHVTKMVARRGAILPQDVSVTPVATPVPPEEQANMWLVEAEISPELEKRLGRKKKRRKRKKEVCPLRPAPELHHSAPVPATSAVPRLPQLPRQKCLVAANAWGTGESCRQGAWTLVSNPFCPEPSPHQDPFLPGASAPRVWAQGRLQGLGSIHSRTNLMEAEILDADSDF

SEQ ID NO: 13 - protein, 786 aa, Homo sapiens
AAARPARGPELPLLGLLLLLLLGDPGRGAASSGNATGPGPRSAGGSARRSAAVTGPPPPLSHCGRAAPCEPLRYNVCLGSVLPYGATSTLLAGDSDSQEEAHGKLVLWSGLRNAPRCWAVIQPLLCAVYMPKCENDRVELPSRTLCQATRGPCAIVERERGWPDFLRCTPDRFPEGCTNEVQNIKFNSSGQCEVPLVRTDNPKSWYEDVEGCGIQCQNPLFTEAEHQDMHSYIAAFGAVTGLCTLFTLATFVADWRNSNRYPAVILFYVNACFFVGSIGWLAQFMDGARREIVCRADGTMRLGEPTSNETLSCVIIFVIVYYALMAGVVWFVVLTYAWHTSFKALGTTYQPLSGKTSYFHLLTWSLPFVLTVAILAVAQVDGDSVSGICFVGYKNYRYRAGFVLAPIGLVLIVGGYFLIRGVMTLFSIKSNHPGLLSEKAASKINETMLRLGIFGFLAFGFVLITFSCHFYDFFNQAEWERSFRDYVLCQANVTIGLPTKQPIPDCEIKNRPSLLVEKINLFAMFGTGIAMSTWVWTKATLLIWRRTWCRLTGQSDDEPKRIKKSKMIAKAFSKRHELLQNPGQELSFSMHTVSHDGPVAGLAFDLNEPSADVSSAWAQHVTKMVARRGAILPQDISVTPVATPVPPEEQANLWLVEAEISPELQKRLGRKKKRRKRKKEVCPLAPPPELHPPAPAPSTIPRLPQLPRQKCLVAAGAWGAGDSCRQGAWTLVSNPFCPEPSPPQDPFLPSAPAPVAWAHGRRQGLGPIHSRTNLMDTELMDADSDF

SEQ ID NO: 14 - protein, 1269 aa, Mus musculus
VDSVGFAEAWRAQFPDSEPPRMELRSVGDIEQELERCKASIRRLEQEVNQERFRMIYLQTLLAKEKKSYDRQRWGFRRAAQPPDGAAEPRASAPRPPPAPADGADPAPVEESEARPDGEGSPSKGRSASARRPAAAASADRDDRGPPTSVAALRSNFEKIRKGPAQPGSADAEKPFYVNVEFHHERGLVKVNDKEVSDRISSLGSQAMQMERKKSQQSAGQGLGEAPRPHYRGRSSESSCGLDGDYEDAELNPRFLKDNLINANGGNRPPWPPLEYQPYQSIYVGGMMVEGEGKSPLLRSQSTSEQEKRLTWPRRSYSPRSFEDSGGGYTPDCSSNENLTSSEEDFSSGQSSRVSPSPTTYRMFRDKSRSPSQNSQQSFDSSSPPTPQCQKRHRQCQVVVSEATIVGVRKTGQIWPSDGDSTFQGEADSSFGTPPGYGCAADQAEEQRRHQDGLPYIDDSPSSSPHLSSKGRGSLASGALDPTKVSELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMETAEKCCQANAQFAEISENLRARSNKDVKDSTTKNSLETLLYKPVDRVTRSTLVLHDLLKHTPSSHPDHSLLQDALRISQNFLSSINEEITPRRQSMTVKKGEHRQLLKDSFMVELVEGARKLRHIFLFTDLLLCTKLKKQSGGKTQQYDCKWYIPLTDLSFQMVDELEALPNIPLVPDEELDALKIKISQIKSDIQREKRANKGSKVMERLRKKLSEQESLLLLMSPSMAFRVHSRNGKSYTFLISSDYERAEWRESIREQQKKCFKSFSLTSVELQMLTNSCVKLQTVHHIPLTINKEDDESPGLYGFLHVIVHSATGFKQSSNLYCTLEVDSFGYFVNKAKTRVYRDTTEPNWNEEFEIELEGSQTLRILCYEKCYNKMKMTKEDGESADKLMGKGQVQLDPQTLQDRDWQRTVIDMNGIEVKLSVKFTSREFSLKRMPSRKQTGVFGVKIAVVTKRERSKVPYIVRQCVEEIERRGMEEVGIYRVSGVATDIQALKAAFDVNNKDVSVMMSEMDVNAIAGTLKLYFRELPEPLFTDEFYPNFAEGIALSDPVAKESCMLNLLLSLPEANLLTFLFLLDHLKRVAEKETVNKMSLHNLATVFGPTLLRPSEKESKLPANPSQPITMTDSWSLEVMSQVQVLLYFLQLEAIPAPDSKRQSILFSTEV

SEQ ID NO: 15 - protein, 1270 aa, Homo sapiens
VDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSNLYCTLEVDSFGYFVNKAKTRVYRDTAEPNWNEEFEIELEGSQTLRILCYEKCYNKTKIPKEDGESTDRLMGKGQVQLDPQALQDRDWQRTVIAMNGIEVKLSVKFNSREFSLKRMPSRKQTGVFGVKIAVVTKRERSKVPYIVRQCVEEIERRGMEEVGIYRVSGVATDIQALKAAFDVNNKDVSVMMSEMDVNAIAGTLKLYFRELPEPLFTDEFYPNFAEGIALSDPVAKESCMLNLLLSLPEANLLTFLFLLDHLKRVAEKEAVNKMSLHNLATVFGPTLLRPSEKESKLPANPSQPITMTDSWSLEVMSQVQVLLYFLQLEAIPAPDSKRQSILFSTEV

SEQ ID NO: 16 - protein, 1226 aa, Homo sapiens
VDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSNLYCTLEVDSFGYFVNKAKTRVYRDTAEPNWNELDPQALQDRDWQRTVIAMNGIEVKLSVKFNSREFSLKRMPSRKQTGVFGVKIAVVTKRERSKVPYIVRQCVEEIERRGMEEVGIYRVSGVATDIQALKAAFDVNNKDVSVMMSEMDVNAIAGTLKLYFRELPEPLFTDEFYPNFAEGIALSDPVAKESCMLNLLLSLPEANLLTFLFLLDHLKRVAEKEAVNKMSLHNLATVFGPTLLRPSEKESKLPANPSQPITMTDSWSLEVMSQVQVLLYFLQLEAIPAPDSKRQSILFSTEV